(19) United States
(12) Patent Application Publication
(10) Pub. No.: US 2024/0000847 A1
(43) Pub. Date: Jan. 4, 2024

(54) METHODS

(71) Applicant: bluebird bio, Inc., Somerville, MA (US)

(72) Inventors: Melissa Bonner, Natick, MA (US); Megan Danielle Hoban, Medford, MA (US)

(21) Appl. No.: 18/035,683

(22) PCT Filed: Nov. 5, 2021
(86) PCT No.: PCT/US2021/058348

§ 371 Date: May 5, 2023

Related U.S. Application Data
(60) Provisional application No. 63/164,181, filed on Mar. 22, 2021.
(60) Provisional application No. 63/110,418, filed on Nov. 6, 2020.

(51) Int. Cl. A61K 35/28; C07K 16/28; A61K 48/00; A61K 31/395; C07K 16/24; A61K 31/675; C12N 15/85

(52) U.S. Cl. CPC A61K 35/28; C07K 16/2803; A61K 48/005; A61K 31/395; C07K 16/243; A61K 31/675; C12N 15/8509; A61K 2039/507

(57) ABSTRACT

The present disclosure provides improved conditioning methods for use in hematopoietic stem cell transplantation and hematopoietic stem cell-based gene therapies.

### CROSS REFERENCE TO RELATED APPLICATIONS

This application claims the benefit under 35 U.S.C. § 119(e) of U.S. Provisional Application No. 63/164,181, filed Mar. 22, 2021, and U.S. Provisional Application No. 63/110,418, filed Nov. 6, 2020, each of which is incorporated by reference herein in its entirety.

### BACKGROUND

### Technical Field

The present invention relates to compositions and methods for conditioning subjects. In particular, the invention also relates to composition and methods for conditioning subjects to receive hematopoietic cell transplantation and gene therapy.

### Description of the Related Art

Hematopoietic stem cell transplantation (HSCT) reconstitutes an individual's hematopoietic system and is routinely used to in cell-based therapies to treat hematological malignancies, hemoglobinopathies, congenital immunodeficiencies, adrenoleukodystrophies, leukodystrophies, HIV/AIDS, and among other diseases, disorders, and conditions. However, the majority of HSCT is performed using non-targeted myeloablative methods that include irradiation (e.g., total body irradiation or TBI) and DNA alkylating/modifying agents that are highly toxic to multiple organ systems and frequently associated with significant morbidity and mortality from both direct toxic effects of chemotherapy as well as opportunistic infections associated with profound immunosuppression.

To fully realize the curative potential of hematopoietic stem cell-based therapies, the development of targeted reduced toxicity conditioning (RTC) regimens that avoid the undesirable toxicities associated with fully myeloablative conditioning while maintaining conditioning efficacy is essential. However, current implementations of antibody based RTC regimens do not efficiently remove host HSCs and ineffectively clear antibodies used to effect the conditioning. Ineffective host HSC removal leads to low levels of therapeutic donor HSC engraftment. Ineffective clearance of RTC antibodies often leads to increased needle-to-needle times for patients awaiting hematopoietic stem cell-based therapies, thereby compounding the problems of effectively bringing RTC into mainstream use for all patients.

### BRIEF SUMMARY

The present disclosure generally relates, in part, to compositions and methods for conditioning subjects to receive hematopoietic cell transplantation and gene therapy.

In various embodiments, a method for hematopoietic stem cell transplantation (HSCT) or HSC-based gene therapy in a subject comprises: administering to the subject, a plurality of first antibodies, and optionally a second antibody, in a dose effective to ablate or decrease hematopoietic stem cells in the bone marrow of the subject; and administering to the subject, donor hematopoietic stem cells.

In certain embodiments, the subject is administered an anti-c-kit antibody and another first antibody that binds an antigen selected from the group consisting of: CD133, CD34, CD33, CD45, CD50, CD123, CD110, and CD90.

In various embodiments, a method for hematopoietic stem cell transplantation (HSCT) or HSC-based gene therapy in a subject comprises: administering to the subject, a first antibody, and optionally a second antibody, in a dose effective to ablate or decrease hematopoietic stem cells in the bone marrow of the subject; administering to the subject, an amount of an endopeptidase effective to degrade or digest and/or inhibit or reduce effector function of the first and second antibodies; and administering to the subject, donor hematopoietic stem cells.

In particular embodiments, the first antibody binds an antigen expressed on a hematopoietic stem cell.

In certain embodiments, the first antibody binds an antigen selected from the group consisting of: c-kit (CD117), CD133, CD34, CD33, CD45, CD50, CD123, CD110, and CD90.

In particular embodiments, the first antibody is conjugated to a cytotoxic agent.

In some embodiments, the first antibody is conjugated to a cytotoxic agent selected from the group consisting of: a toxin, a radioisotope, an RNA polymerase II inhibitor and/or RNA polymerase III inhibitor, and a DNA-damaging agent.

In particular embodiments, the second antibody is an anti-CD47 antibody or an anti-SIRPα antibody.

In some embodiments, the endopeptidase is a cysteine protease or a thiol protease.

In various embodiments, the endopeptidase is isolated from Streptococcus pyogenes, Streptococcus equi, Streptococcus agalactiae, Streptococcus pseudoporcinus, Streptococcus suis, Streptococcus porcinus, Mycoplasma canis.

In further embodiments, the endopeptidase comprises an IgG degrading enzyme (IgdE).

In certain embodiments, the endopeptidase comprises an IgdE selected from the group consisting of: an Immunoglobulin G-degrading enzyme of S. pyogenes (IdeS), Immunoglobulin G-degrading enzyme of S. equi ssp. zooepidemicus (IdeZ), an Immunoglobulin G-degrading enzyme of M. canis (IdeMC), and variants thereof.

In various embodiments, a method for hematopoietic stem cell transplantation (HSCT) or adoptive cell therapy (ACT) in a subject comprises: administering to the subject, a first antibody, wherein the first antibody binds an antigen expressed on hematopoietic stem cells; administering to the subject, a second antibody, wherein the second antibody blocks CD47 binding to SIRPα; wherein the first antibody and the second antibody are each administered in a dose effective to ablate hematopoietic stem cells in the bone marrow of the subject; administering to the subject, an amount of an endopeptidase effective to degrade or digest and/or inhibit or reduce effector function of the first and second antibodies; and administering to the subject, donor hematopoietic stem cells.

In particular embodiments, the first antibody binds an antigen selected from the group consisting of: c-kit (CD117), CD133, CD34, CD33, CD45, CD50, CD123, CD110, and CD90.

In some embodiments, the first antibody binds CD117 (c-kit).

In further embodiments, the first antibody binds CD133.

In particular embodiments, the first antibody binds CD34.

In particular embodiments, the first antibody binds CD33.

In additional embodiments, the first antibody binds CD45

In particular embodiments, the first antibody binds CD50.

In certain embodiments, the first antibody binds CD123.

In some embodiments, the first antibody binds CD110.

In particular embodiments, the first antibody binds CD90.

In further embodiments, the first antibody is conjugated to a cytotoxic agent.

In certain embodiments, the first antibody is conjugated to a cytotoxic agent selected from the group consisting of: a toxin, a radioisotope, an RNA polymerase II inhibitor and/or RNA polymerase III inhibitor, and a DNA-damaging agent.

In various embodiments, the second antibody is an anti-CD47 antibody or an anti-SIRPα antibody.

In additional embodiments, the second antibody is an anti-CD47 antibody.

In further embodiments, the second antibody is an anti-SIRPα antibody.

In particular embodiments, the endopeptidase is a cysteine protease or a thiol protease.

In some embodiments, the endopeptidase is isolated from Streptococcus pyogenes, Streptococcus equi, Streptococcus agalactiae, Streptococcus pseudoporcinus, Streptococcus suis, Streptococcus porcinus, Mycoplasma canis.

In certain embodiments, the endopeptidase comprises an IgG degrading enzyme (IgdE).

In additional embodiments, the endopeptidase comprises an IgdE selected from the group consisting of: an Immunoglobulin G-degrading enzyme of S. pyogenes (IdeS), Immunoglobulin G-degrading enzyme of S. equi ssp. zooepidemicus (IdeZ), an Immunoglobulin G-degrading enzyme of M. canis (IdeMC), and variants thereof.

In various embodiments, a method for hematopoietic stem cell transplantation (HSCT) or adoptive cell therapy (ACT) in a subject comprises: administering a first antibody to the subject, wherein the first antibody is an anti-CD117 antibody; administering a second antibody to the subject, wherein the second antibody is an anti-CD47 or anti-SIRPα antibody blocks CD47 binding to SIRPα; wherein the first antibody and the second antibody are each administered in a dose effective to ablate hematopoietic stem cells in the bone marrow of the subject; administering to the subject, an amount of an endopeptidase effective to degrade or digest and/or inhibit or reduce effector function of the first antibody and the second antibody; and administering to the subject, donor hematopoietic stem cells.

In further embodiments, the first antibody is conjugated to a cytotoxic agent.

In particular embodiments, the first antibody is conjugated to a cytotoxic agent selected from the group consisting of: a toxin, a radioisotope, an RNA polymerase II inhibitor and/or RNA polymerase III inhibitor, and a DNA-damaging agent.

In additional embodiments, the second antibody is an anti-CD47 antibody.

In some embodiments, the second antibody is an anti-SIRPα antibody.

In further embodiments, the endopeptidase is a cysteine protease or a thiol protease.

In particular embodiments, the endopeptidase is isolated from Streptococcus pyogenes, Streptococcus equi, Streptococcus agalactiae, Streptococcus pseudoporcinus, Streptococcus suis, Streptococcus porcinus, Mycoplasma canis.

In additional embodiments, the endopeptidase comprises an IgG degrading enzyme (IgdE).

In certain embodiments, the endopeptidase comprises an IgdE selected from the group consisting of: an Immunoglobulin G-degrading enzyme of S. pyogenes (IdeS), Immunoglobulin G-degrading enzyme of S. equi ssp. zooepidemicus (IdeZ), an Immunoglobulin G-degrading enzyme of M. canis (IdeMC), and variants thereof.

In various embodiments, a method for hematopoietic stem cell transplantation (HSCT) or adoptive cell therapy (ACT) in a subject comprises: administering a first antibody to the subject, wherein the first antibody is an anti-CD117 antibody; administering a second antibody to the subject, wherein the first antibody is an anti-CD47 antibody that blocks CD47 binding to SIRPα; wherein the first antibody and the second antibody are each administered in a dose effective to ablate hematopoietic stem cells in the bone marrow of the subject; administering to the subject, an amount of a cysteine protease effective to degrade or digest and/or inhibit or reduce effector function of the first and second antibodies, wherein the protease or glycosidase is a cysteine protease; and administering to the subject, donor hematopoietic stem cells.

In some embodiments, the first antibody is conjugated to a cytotoxic agent.

In particular embodiments, the first antibody is conjugated to a cytotoxic agent selected from the group consisting of: a toxin, a radioisotope, an RNA polymerase II inhibitor and/or RNA polymerase III inhibitor, and a DNA-damaging agent.

In additional embodiments, the cysteine protease is isolated from Streptococcus pyogenes, Streptococcus equi, Streptococcus agalactiae, Streptococcus pseudoporcinus, Streptococcus suis, Streptococcus porcinus, Mycoplasma canis.

In further embodiments, the cysteine protease comprises an IgG degrading enzyme (IgdE).

In certain embodiments, the cysteine protease comprises an IgdE selected from the group consisting of: an Immunoglobulin G-degrading enzyme of S. pyogenes (IdeS), Immunoglobulin G-degrading enzyme of S. equi ssp. zooepidemicus (IdeZ), an Immunoglobulin G-degrading enzyme of M. canis (IdeMC), and variants thereof.

In particular embodiments, the first antibody and the second antibody are administered to the subject at about the same time.

In additional embodiments, the first antibody is administered to the subject before the second antibody is administered to the subject.

In particular embodiments, the first antibody is administered to the subject after the second antibody is administered to the subject.

In some embodiments, the endopeptidase is administered to the subject after both the first antibody and the second antibody have been administered to the subject.

In certain embodiments, the endopeptidase is administered to the subject at least 3 days, at least 4 days, at least 5 days, at least 6 days, at least 7 days, at least 8 days, at least 9 days, at least 10 days, at least 11 days, at least 12 days, at least 13 days, or at least 14 days after the day that the last of the first antibody and the second antibody have been administered to the subject.

In further embodiments, the donor hematopoietic stem cells are administered to the subject after the endopeptidase is administered to the subject.

In various embodiments, the donor hematopoietic stem cells are administered to the subject at least 3 days, at least 4 days, at least 5 days, at least 6 days, at least 7 days, at least 8 days, at least 9 days, at least 10 days, at last 11 days, at least 12 days, at least 13 days, or at least 14 days after the endopeptidase is administered to the subject.

In additional embodiments, the donor hematopoietic stem cells are administered to the subject no more than 21 days, no more than 20 days, no more than 19 days, no more than 18 days, no more than 17 days, no more than 16 days, no more than 15 days, no more than 14 days, no more than 13 days, no more than 12 days, no more than 11 days, no more than 10 days, no more than 9 days, no more than 8 days, no more than 7 days, no more than 6 days, or no more than 5 days after the endopeptidase is administered to the subject.

In some embodiments, the donor hematopoietic stem cells are allogenic to the subject.

In particular embodiments, the donor hematopoietic stem cells are autologous to the subject.

In further embodiments, the donor hematopoietic stem cells comprise a vector comprising a polynucleotide that encodes a therapeutic polypeptide.

In certain embodiments, the donor hematopoietic stem cells comprise one or more genome edits.

In particular embodiments, the donor hematopoietic stem cells comprise a vector comprising a polynucleotide that encodes a therapeutic polypeptide and one or more genome edits.

In some embodiments, the donor hematopoietic stem cells comprise a cell-based gene therapy.

In additional embodiments, the subject has a disease amenable to treatment with a cell-based gene therapy.

In various embodiments, the subject has a disease selected from the group consisting of an autoimmune disease, a hemoglobinopathy, a leukodystropy, a lysosomal storage disease, and a neurogenerative disease.

In further embodiments, the subject has a disease selected from the group consisting of ADA-SCID, Artemis protein deficiency, Batten disease, β-thalassemia, sickle cell disease, cerebral ALD, chronic granulomatous diseases, Fabry disease, Fanconi anemia, Gaucher disease, Hemophilia A, Hemophilia B, Leukocyte adhesion deficiency, metachromatic leukodystrophy, MPS I (Hurler syndrome), MPS II (Hunter's syndrome), MPS III (Sanfilippo Syndrome), MPS IV-A (Morquio A Syndrome), MPS IV-B (Morquio B Syndrome), MPS VI (Maroteaux-Lamy Syndrome), Pompe Disease (acid alpha-glucosidase); RAG deficiency, Wiskott Aldrich syndrome, X-linked lymphoproliferative syndrome, and X-Linked Severe Combined Immunodeficiency (X-SCID).

In additional embodiments, the subject has ADA-SCID and is administered a cell-based gene therapy comprising hematopoietic stem cells comprising a vector that comprises a polynucleotide encoding adenosine deaminase.

In particular embodiments, the subject has Artemis protein deficiency and is administered a cell-based gene therapy comprising hematopoietic stem cells comprising a vector that comprises a polynucleotide encoding DCLRE1C.

In some embodiments, the subject has Batten Disease and is administered a cell-based gene therapy comprising hematopoietic stem cells comprising a vector that comprises a polynucleotide encoding CLN1, CLN2, CLN3, CLN4, CLN5, CLN6, CLN7, CLN8, or CLN10.

In certain embodiments, the subject has CALD and is administered a cell-based gene therapy comprising hematopoietic stem cells comprising a vector that comprises a polynucleotide encoding ATP-binding cassette, sub-family D, member 1 (ABCD1).

In various embodiments, the subject has a chronic granulomatous disease and is administered a cell-based gene therapy comprising hematopoietic stem cells comprising a vector that comprises a polynucleotide encoding cytochrome b-245 alpha chain, cytochrome b-245 beta chain, neutrophil cytosolic factor 1, neutrophil cytosolic factor 2, or neutrophil cytosolic factor 4.

In further embodiments, the subject has Fabry disease and is administered a cell-based gene therapy comprising hematopoietic stem cells comprising a vector that comprises a polynucleotide encoding alpha-galactosidase A.

In particular embodiments, the subject has Fanconi anemia and is administered a cell-based gene therapy comprising hematopoietic stem cells comprising a vector that comprises a polynucleotide encoding FANCA, FANCC, or FANCG.

In additional embodiments, the subject has Gaucher disease and is administered a cell-based gene therapy comprising hematopoietic stem cells comprising a vector that comprises a polynucleotide encoding glucocerebrosidase.

In various embodiments, the subject has Hemophilia A and is administered a cell-based gene therapy comprising hematopoietic stem cells comprising a vector that comprises a polynucleotide encoding Factor VIII.

In some embodiments, the subject has Hemophilia B and is administered a cell-based gene therapy comprising hematopoietic stem cells comprising a vector that comprises a polynucleotide encoding Factor IX.

In further embodiments, the subject has Leukocyte adhesion deficiency and is administered a cell-based gene therapy comprising hematopoietic stem cells comprising a vector that comprises a polynucleotide encoding CD18.

In particular embodiments, the subject has MLD and is administered a cell-based gene therapy comprising hematopoietic stem cells comprising a vector that comprises a polynucleotide encoding arylsulfatase A.

In additional embodiments, the subject has MPS I and is administered a cell-based gene therapy comprising hematopoietic stem cells comprising a vector that comprises a polynucleotide encoding alpha-L-iduronidase.

In certain embodiments, the subject has MPS II and is administered a cell-based gene therapy comprising hematopoietic stem cells comprising a vector that comprises a polynucleotide encoding iduronate 2-sulfatase.

In various embodiments, the subject has MPS III and is administered a cell-based gene therapy comprising hematopoietic stem cells comprising a vector that comprises a polynucleotide encoding N-sulfoglucosamine sulfohydrolase.

In particular embodiments, the subject has MPS IV-A and is administered a cell-based gene therapy comprising hematopoietic stem cells comprising a vector that comprises a polynucleotide encoding galactosamine-6 sulfatase.

In some embodiments, the subject has MPS IV-B and is administered a cell-based gene therapy comprising hematopoietic stem cells comprising a vector that comprises a polynucleotide encoding beta-galactosidase.

In further embodiments, the subject has MPS VI and is administered a cell-based gene therapy comprising hematopoietic stem cells comprising a vector that comprises a polynucleotide encoding N-acetylgalactosamine-4-sulphatase.

In certain embodiments, the subject has Pompe Disease and is administered a cell-based gene therapy comprising hematopoietic stem cells comprising a vector that comprises a polynucleotide encoding acid alpha-glucosidase

In various embodiments, the subject has a RAG deficiency and is administered a cell-based gene therapy comprising hematopoietic stem cells comprising a vector that comprises a polynucleotide encoding RAG1 or RAG2.

In particular embodiments, the subject has Wiskott-Aldrich syndrome and is administered a cell-based gene therapy comprising hematopoietic stem cells comprising a vector that comprises a polynucleotide encoding Wiskott-Aldrich syndrome protein.

In additional embodiments, the subject has X-SCID and is administered a cell-based gene therapy comprising hematopoietic stem cells comprising a vector that comprises a polynucleotide encoding interleukin 2 receptor gamma.

In some embodiments, the subject has a hemoglobinopathy and is administered a cell-based gene therapy comprising hematopoietic stem cells comprising a vector that comprises a polynucleotide encoding a globin protein.

In particular embodiments, the subject has β-thalassemia and is administered a cell-based gene therapy comprising hematopoietic stem cells comprising a vector that comprises a polynucleotide encoding a β-globin protein, a δ-globin protein, a γ-globin protein, an anti-sickling globin, a βA-T87Q-globin protein, a βA-G16D/E22A/T87Q-globin protein, or a βA-T87Q/K95E/K120E-globin protein.

In various embodiments, the subject has sickle cell disease and is administered a cell-based gene therapy comprising hematopoietic stem cells comprising a vector that comprises a polynucleotide encoding a β-globin protein, a δ-globin protein, a γ-globin protein, an anti-sickling globin, a βA-T87Q-globin protein, a βA-G16D/E22A/T87Q-globin protein, or a βA-T87Q/K95E/K120E-globin protein.

In particular embodiments, method contemplated herein comprise a further step of administering one or more HSC mobilization agents to the subject before a subject is administered a first antibody and/or second antibody.

In certain embodiments, the one or more HSC mobilization agents is selected from the group consisting of: GM-CSF, G-CSF (filgrastim), pegylated G-CSF (pegfilgrastim), glycosylated G-CSF (lenograstim), AMD3100 (plerixafor), macrophage inflammatory protein 1α (MIP1α), CCL3, SDF-1α peptide analogs (CTCE-0021, CTCE-0214), Met-SDF-1β, IL-8, GRO proteins (GRO-β (CXCL2)), SB-251353, and suitable combinations thereof.

In some embodiments, the one or more HSC mobilization agents is G-CSF (filgrastim) and/or AMD3100 (plerixafor).

In particular embodiments, the one or more HSC mobilization agents is G-CSF (filgrastim) and AMD3100 (plerixafor).

In particular embodiments, method contemplated herein comprise a further step of administering one or more chemotherapeutic agents to a subject before the subject is administered a first antibody and/or second antibody, and in an amount effective to increase macrophage activation and recruitment to the bone marrow of the subject.

In some embodiments, the one or more chemotherapeutic agents is selected from the group consisting of: cyclophosphamide, busulphan, treosulphan, melphalan, thiotepa, carboplatin, carmustine, etoposide, cytosine arabinoside (AraC), and fludarabine.

In certain embodiments, the one or more chemotherapeutic agents is cyclophosphamide.

### BRIEF DESCRIPTION OF SEVERAL VIEWS OF THE DRAWINGS

FIG. 1 shows IdeZ cleavage of murine anti-CD117 antibody inhibits mIgG3 binding to CD117 expressing EML cells.

FIG. 2A shows increased macrophage recruitment in bone marrow of cyclophosphamide treated mice compared to mice treated with vehicle (top panel). FIG. 2A also shows increased macrophage activation in macrophages recruited to bone marrow of cyclophosphamide treated mice compared to mice treated with vehicle (bottom panel).

FIG. 2B shows increased macrophage staining in bone marrow smears in cyclophosphamide treated mice (Ctx, dosed at 2×100 mg/kg) compared to mice treated with vehicle.

FIG. 3 shows that mice treated with a combination of cyclophosphamide and anti-CD117 and anti-CD47 antibodies had increased stem cell depletion in the bone marrow (left panel) and fewer colony forming units (CFUs) from bone marrow cells (right panel) compared to mice treated with vehicle or anti-CD117 and anti-CD47 antibodies alone.

### DETAILED DESCRIPTION

### A. Overview

The present disclosure generally relates to, in part, improved reduced toxicity conditioning (RTC) methods.

For decades, hematopoietic stem cell transplantation (HSCT) has served as a powerful treatment modality, enabling reconstitution of a patient's hematopoietic system with healthy donor or genetically modified hematopoietic stem cells (HSCs). Although HSCT often results in life-long benefits and potentially cures many malignant and non-malignant blood and immune diseases, it is estimated that less than 25% of patients that could benefit from HSCT undergo transplantation. The dramatic underutilization of HSCT is primarily due to high rates of morbidity/mortality from harsh conditioning methods that are currently necessary to enable donor HSC engraftment by making “space” in host bone marrow (BM) for donor HSC engraftment.

Classical conditioning regimens using total body irradiation (TBI) or chemotherapy, e.g., a combination of myeloablative doses of busulfan (Bu) and cyclophosphamide, lead to both detrimental short-term and long-term complications including multi-organ damage, mucositis, need for frequent red blood cell and platelet transfusions, infertility, and malignancies. Harsh conditioning regimens can also lead to profound and prolonged immune ablation, which predisposes patients to serious and sometimes fatal opportunistic infections necessitating extended hospitalizations and exposure to toxic side effects of anti-infective agents.

Newer chemotherapy regimens have been described as reduced intensity conditioning (RIC). RIC refers to methods that are not as intense and thus, less effective than myeloablative conditioning. For example, substituting treosulfan (Treo) for busulfan, in combination with fludarabine (Flu), is undoubtedly less toxic, but the combination of Treo Flu does not consistently achieve full complete conditioning. Thus, reduced toxicity conditioning (RTC) is preferred, i.e., where conditioning is effective as myeloablative conditioning but does not carry the toxicities normally associated with them.

The methods contemplated in the present disclosure solve the problems associated with suboptimal RTC used in hematopoietic stem cells transplants (HSCTs) and HSC-based gene therapy.

One solution to suboptimal RTC contemplated in particular embodiments comprises increasing RTC antibody-dependent phagocytosis. Without wishing to be bound by any particular theory, it is contemplated that subjects can be administered low or sub-ablative doses of chemotherapeutics to increase macrophage recruitment, and/or activation, to the bone marrow or hematopoietic stem cell niche and increase the efficacy of, and/or enhance the RTC methods contemplated herein by increasing antibody dependent phagocytosis of HSCs targeted by RTC antibodies.

In various embodiments, a method for HSCT or HSC-based gene therapy in a subject comprises administering an agent in an amount effective to increase macrophage recruitment, and/or activation, to the bone marrow of a subject; administering an antibody-based RTC regimen to the subject; and administering donor hematopoietic stem cells to the subject.

In particular embodiments, a method for HSCT or HSC-based gene therapy in a subject comprises administering an agent in an amount effective to increase macrophage recruitment, and/or activation, to the bone marrow of a subject; administering an antibody-based RTC regimen to the subject; administering an enzyme, e.g., a protease or glycosidase, that degrades the one or more antibodies used in the RTC regimen; and administering donor hematopoietic stem cells to the subject.

Another solution to suboptimal RTC contemplated in particular embodiments comprises increasing RTC antibody access to the host HSCs. Without wishing to be bound by any particular theory, it is contemplated that HSC mobilization clears the HSC niche and increases the ability of RTC antibodies to target HSCs thereby increasing the efficacy of, and/or enhancing the RTC methods contemplated herein.

In various embodiments, a method for HSCT or HSC-based gene therapy in a subject comprises administering one or more HSC mobilization agents to the subject; administering an antibody-based RTC regimen to the subject; and administering donor hematopoietic stem cells to the subject.

In particular embodiments, a method for HSCT or HSC-based gene therapy in a subject comprises administering one or more HSC mobilization agents to the subject; administering an antibody-based RTC regimen to the subject; administering an enzyme, e.g., a protease or glycosidase, that degrades the one or more antibodies used in the RTC regimen; and administering donor hematopoietic stem cells to the subject. In various embodiments, a method for HSCT or HSC-based gene therapy in a subject comprises a step of administering an antibody-based RTC regimen to a subject, a step of administering an enzyme, e.g., a protease or glycosidase, that degrades the one or more antibodies used in the RTC regimen; and a step of administering donor hematopoietic stem cells to the subject.

The antibody-based RTC regimen may comprise a single antibody, one or more antibodies, or one, two, three, four, or five or more antibodies, or antigen binding fragments thereof. In particular embodiments, an antibody-based RTC regimen comprises administering one or more antibodies, that bind one or more antigens expressed on an HSC.

In particular embodiments, an RTC antibody that binds an antigen expressed on an HSC is conjugated to a cytotoxic agent.

In particular embodiments, an antibody-based conditioning regimen comprises administration of one or more antibodies that bind one or more antigens expressed on an HSC (a first antibody(ies)) and administration of an antibody that binds an antigen that provides a “don't eat me” signal (a second antibody), which normally protects the cells from being engulfed by macrophages. Without wishing to be bound by any particular theory, blocking the don't eat me signal, enables macrophages to engulf the target cells bound by the first antibody. In particular embodiments, the second antibody binds to a protein expressed on the HSC to block the don't eat me signal. In other particular embodiments, the second antibody binds a protein expressed on a macrophage, thereby blocking the don't eat me signal.

Methods contemplated herein, comprise an antibody-based RTC regimen followed by administration of a protease or glycosidase effective to degrade or digest and/or inhibit or reduce effector function of the first and second RTC antibodies. In certain embodiments, the protease or glycosidase is a cysteine protease or a thiol protease. In certain embodiments, the protease comprises an IgG degrading enzyme (IgdE).

Methods contemplated herein, further administering a population of donor cells comprising HSCs to a subject. In particular embodiments, a population of donor cells comprises CD34⁺ selected hematopoietic stem cells. In particular embodiments, donor cells may comprise one or more genetic modifications and/or comprise one or more gene therapy vectors or provectors. In other particular embodiments, donor cells are not genetically modified.

In certain embodiments, the donor cells are allogeneic to the donor. In other certain embodiments, the donor cells are autologous to the donor. In yet other certain embodiments, the donor cells are xenogenic to the donor.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and C C Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, 10%, ±9%, ±8%, ±7%, ±6%, ±5%, ±4%, ±3%, ±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

A “subject,” as used herein, includes any animal that exhibits a symptom of a disease, disorder, or condition that can be treated with the hematopoietic stem cell transplantation methods or HSC-based gene therapy methods contemplated herein.

Suitable subjects (e.g., patients) include laboratory animals (such as mouse, rat, rabbit, or guinea pig), farm animals, and domestic animals or pets (such as a cat or dog). Non-human primates and human patients are preferred subjects in particular embodiments.

As used herein, the term “patient” refers to a subject that has been diagnosed with disease, disorder, or condition that can be treated with the hematopoietic stem cell transplantation methods or HSC-based gene therapy methods contemplated elsewhere herein.

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

The term “hematopoietic stem cell” or “HSC” refers to multipotent stem cells that give rise to the all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827). Hematopoietic stem cells markers include but are not limited to c-kit (CD117), CD133, CD34, CD45, and CD90. When transplanted into lethally irradiated animals or humans, hematopoietic stem cells can repopulate the erythroid, neutrophil-macrophage, megakaryocyte and lymphoid hematopoietic cell pool.

An “antibody” refers to a binding agent that is a polypeptide comprising at least a light chain or heavy chain immunoglobulin variable region which specifically recognizes and binds an epitope of an antigen, such as a lipid, carbohydrate, polysaccharide, glycoprotein, peptide, or nucleic acid containing an antigenic determinant, such as those recognized by an immune cell.

An “epitope” or “antigenic determinant” refers to the region of an antigen to which a binding agent binds.

Antibodies include antigen binding fragments thereof, such as a Camel Ig, a Llama Ig, an Alpaca Ig, Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, a camelid VHH, Nanobody) and portions of full length antibodies responsible for antigen binding. Antibodies also include: polyclonal and monoclonal antibodies and antigen binding fragments thereof; murine antibodies, camelid antibodies, and human antibodies, and antigen binding fragments thereof; and chimeric antibodies, heteroconjugate antibodies, and humanized antibodies, and antigen binding fragments thereof. See also, Pierce Catalog and Handbook, 1994-1995 (Pierce Chemical Co., Rockford, IL); Kuby, J., Immunology, 3rd Ed., W. H. Freeman & Co., New York, 1997.

By “enhance” or “promote,” or “increase” or “expand” refers generally to the ability of a composition contemplated herein to produce, elicit, or cause a greater physiological response (i.e., downstream effects) compared to the response caused by either vehicle or a control molecule/composition. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. A “decrease” or “reduced” amount is typically a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, or a control composition.

By “maintain,” or “preserve,” or “maintenance,” or “no change,” or “no substantial change,” or “no substantial decrease” refers generally to the ability of a composition contemplated herein to produce, elicit, or cause a substantially similar or comparable physiological response (i.e., downstream effects) in a cell, as compared to the response caused by either vehicle, or a control molecule/composition. A comparable response is one that is not significantly different or measurable different from the reference response.

As used herein, the term “genetically engineered” or “genetically modified” refers to the chromosomal or extrachromosomal addition of extra genetic material in the form of DNA or RNA to the total genetic material in a cell. Genetic modifications may be targeted or non-targeted to a particular site in a cell's genome. In one embodiment, genetic modification is site specific. In one embodiment, genetic modification is not site specific.

As used herein, the term “genome editing” refers to the substitution, deletion, and/or introduction of genetic material at a target site in the cell's genome, which restores, corrects, disrupts, and/or modifies expression and/or function of a gene or gene product. Genome editing contemplated in particular embodiments comprises introducing one or more nuclease variants into a cell to generate DNA lesions at or proximal to a target site in the cell's genome, optionally in the presence of a donor repair template.

As used herein, the term “gene therapy” refers to the introduction of extra genetic material into the total genetic material in a cell that restores, corrects, or modifies expression of a gene or gene product, or for the purpose of expressing a therapeutic polypeptide. In particular embodiments, introduction of genetic material into the cell's genome by genome editing that restores, corrects, disrupts, or modifies expression of a gene or gene product, or for the purpose of expressing a therapeutic polypeptide is considered gene therapy.

Additional definitions are set forth throughout this disclosure.

### C. Methods

Antibody-based methods for existing reduced toxicity conditioning (RTC) regimens in hematopoietic stem cell transplant suffer from incomplete access to target cells, target cell ablation, and/or inefficient removal of conditioning agents. Current methods prolong needle-to-needle times and reduce the chances of a successful graft and ultimately decrease the likelihood of a successful therapeutic outcome.

In various embodiments, a method for a hematopoietic stem cell transplantation (HSCT) or an HSC-based gene therapy in a subject comprises administering a first antibody that binds an antigen expressed on an HSC in a dose effective to ablate, eradicate, eliminate, decrease, or induce cell death of the subject's hematopoietic stem cells; administering a protease or glycosidase that degrades or digests and/or inhibits or reduces effector function of the first antibody(ies); and administering a population of donor cells comprising hematopoietic stem cells to the subject. In particular embodiments, the donor cells comprise CD34⁺ selected hematopoietic stem cells. In particular embodiments, the donor cells comprise one or more genetic modifications.

In various embodiments, a method for a hematopoietic stem cell transplantation (HSCT) or an HSC-based gene therapy in a subject comprises administering a first antibody that binds to an antigen expressed on an HSC and a second antibody that binds to another antigen expressed on an HSC or an antigen expressed on a macrophage, wherein the second antibody enables macrophages to recognize and engulf the HSCs bound by the first antibody; administering an endopeptidase that degrades or digests and/or inhibits or reduces effector function of the first antibody and the second antibody; and administering donor cells comprising hematopoietic stem cells to the subject. In particular embodiments, the donor cells comprise CD34⁺ selected hematopoietic stem cells. In particular embodiments, the donor cells comprise one or more genetic modifications.

In various embodiments, a method for a hematopoietic stem cell transplantation (HSCT) or an HSC-based gene therapy in a subject comprises administering one or more antibodies (a first antibody) that bind to one or more antigens expressed on an HSC in a dose effective to ablate, eradicate, eliminate, decrease, or induce cell death of the subject's hematopoietic stem cells; administering a protease or glycosidase that degrades or digests and/or inhibits or reduces effector function of the first antibody(ies); and administering a population of donor cells comprising hematopoietic stem cells to the subject. In particular embodiments, the donor cells comprise CD34⁺ selected hematopoietic stem cells. In particular embodiments, the donor cells comprise one or more genetic modifications.

In various embodiments, a method for a hematopoietic stem cell transplantation (HSCT) or an HSC-based gene therapy in a subject comprises administering one or more antibodies (a first antibody) that bind to one or more antigens expressed on an HSC and a second antibody that binds to another antigen expressed on an HSC or an antigen expressed on a macrophage, wherein the second antibody enables macrophages to recognize and engulf the HSCs bound by the first antibody(ies); administering an endopeptidase that degrades or digests and/or inhibits or reduces effector function of the first antibody(es) and the second antibody; and administering donor cells comprising hematopoietic stem cells to the subject. In particular embodiments, the donor cells comprise CD34⁺ selected hematopoietic stem cells. In particular embodiments, the donor cells comprise one or more genetic modifications.

In various embodiments, a method for a hematopoietic stem cell transplantation (HSCT) or an HSC-based gene therapy in a subject comprises administering one or more HSC mobilizing agents; administering a first antibody that binds to an antigen expressed on an HSC and a second antibody that binds to another antigen expressed on an HSC or an antigen expressed on a macrophage, wherein the second antibody enables macrophages to recognize and engulf the HSCs bound by the first antibody; and administering donor cells comprising hematopoietic stem cells to the subject. In particular embodiments, the donor cells comprise CD34⁺ selected hematopoietic stem cells. In particular embodiments, the donor cells comprise one or more genetic modifications.

In various embodiments, a method for a hematopoietic stem cell transplantation (HSCT) or an HSC-based gene therapy in a subject comprises administering one or more HSC mobilizing agents; administering a first antibody that binds to an antigen expressed on an HSC and a second antibody that binds to another antigen expressed on an HSC or an antigen expressed on a macrophage, wherein the second antibody enables macrophages to recognize and engulf the HSCs bound by the first antibody; administering an endopeptidase that degrades or digests and/or inhibits or reduces effector function of the first antibody and the second antibody; and administering donor cells comprising hematopoietic stem cells to the subject. In particular embodiments, the donor cells comprise CD34⁺ selected hematopoietic stem cells. In particular embodiments, the donor cells comprise one or more genetic modifications.

In various embodiments, a method for a hematopoietic stem cell transplantation (HSCT) or an HSC-based gene therapy in a subject comprises administering one or more HSC mobilizing agents; administering a first antibody that binds to an antigen expressed on an HSC and a second antibody that binds to another antigen expressed on an HSC or an antigen expressed on a macrophage, wherein the second antibody enables macrophages to recognize and engulf the HSCs bound by the first antibody; administering an endopeptidase that degrades or digests and/or inhibits or reduces effector function of the first antibody and the second antibody; and administering donor cells comprising hematopoietic stem cells to the subject. In particular embodiments, the donor cells comprise CD34⁺ selected hematopoietic stem cells. In particular embodiments, the donor cells comprise one or more genetic modifications.

In various embodiments, a method for a hematopoietic stem cell transplantation (HSCT) or an HSC-based gene therapy in a subject comprises administering one or more chemotherapeutic agents in an amount effective to increase macrophage activation and recruitment to the bone marrow; administering a first antibody that binds to an antigen expressed on an HSC and a second antibody that binds to another antigen expressed on an HSC or an antigen expressed on a macrophage, wherein the second antibody enables macrophages to recognize and engulf the HSCs bound by the first antibody; and administering donor cells comprising hematopoietic stem cells to the subject. In particular embodiments, the donor cells comprise CD34⁺ selected hematopoietic stem cells. In particular embodiments, the donor cells comprise one or more genetic modifications.

In various embodiments, a method for a hematopoietic stem cell transplantation (HSCT) or an HSC-based gene therapy in a subject comprises administering one or more chemotherapeutic agents in an amount effective to increase macrophage activation and recruitment to the bone marrow; administering a first antibody that binds to an antigen expressed on an HSC and a second antibody that binds to another antigen expressed on an HSC or an antigen expressed on a macrophage, wherein the second antibody enables macrophages to recognize and engulf the HSCs bound by the first antibody; administering an endopeptidase that degrades or digests and/or inhibits or reduces effector function of the first antibody and the second antibody; and administering donor cells comprising hematopoietic stem cells to the subject. In particular embodiments, the donor cells comprise CD34⁺ selected hematopoietic stem cells. In particular embodiments, the donor cells comprise one or more genetic modifications.

In various embodiments, a method for a hematopoietic stem cell transplantation (HSCT) or an HSC-based gene therapy in a subject comprises administering one or more chemotherapeutic agents in an amount effective to increase macrophage activation and recruitment to the bone marrow; administering a first antibody that binds to an antigen expressed on an HSC and a second antibody that binds to another antigen expressed on an HSC or an antigen expressed on a macrophage, wherein the second antibody enables macrophages to recognize and engulf the HSCs bound by the first antibody; administering an endopeptidase that degrades or digests and/or inhibits or reduces effector function of the first antibody and the second antibody; and administering donor cells comprising hematopoietic stem cells to the subject. In particular embodiments, the donor cells comprise CD34⁺ selected hematopoietic stem cells. In particular embodiments, the donor cells comprise one or more genetic modifications.

1. First Antibody

Methods contemplated herein comprise administration of a first antibody that binds to a protein expressed on a hematopoietic stem cell. In some embodiments, one or more first antibodies are administered to a subject, i.e., one or more antibodies that bind to a protein expressed on a hematopoietic stem cell for the purpose of targeting or removing the cell from the niche. In particular embodiments, a method comprises administering an anti-CD117 antibody and another first antibody to the subject. Without wishing to be bound by any particular theory, it is contemplated that a first antibody conjugated to a cytotoxic agent will lead to internalization of the agent by the HSC and cell death. It is further contemplated that when a method comprises administration of a first antibody and a second antibody, that the first antibody can be conjugated to a cytotoxic agent in particular embodiments, but in preferred embodiments, the first antibody is not conjugated to a cytotoxic agent so as not to interfere with recognition and engulfment of the HSCs by macrophages. Efficient removal of resident HSCs in the bone marrow makes room for donor HSCs to engraft.

In particular embodiments, the first antibody binds an antigen expressed on a hematopoietic stem cell. In particular embodiments, the antigen is selectively expressed on a hematopoietic cell or specifically expressed on a hematopoietic cell. In particular embodiments, the antigen is selectively expressed on a hematopoietic stem cell and/or a hematopoietic progenitor cell or specifically expressed on a hematopoietic stem cell or hematopoietic progenitor cell. In particular embodiments, the antigen is selectively expressed on a hematopoietic stem cell or specifically expressed on a hematopoietic stem cell. In particular embodiments, the antigen is selected from the group consisting of: c-kit (CD117), CD133, CD123, CD110, CD34, CD33, CD45, CD50, and CD90.

In particular embodiments, there are a plurality of first antibodies that are used. In some embodiments, one or more antibodies binding one or more antigens expressed on a hematopoietic stem cell are administered to the subject. In particular embodiments, one or more first antibodies that bind one or more of c-kit (CD117), CD133, CD123, CD110, CD34, CD50, CD33, CD45, and CD90 are administered to the subject.

In particular embodiments, the subject is administered an anti-CD117 antibody and another first antibody that binds one or more of c-kit (CD117), CD133, CD123, CD110, CD34, CD50, CD33, CD45, and CD90.

In a preferred embodiment, the first antibody binds to CD117.

In particular embodiments, the first antibody is conjugated to a cytotoxic agent.

In certain illustrative embodiments, the first antibody is conjugated to a cytotoxic agent selected from the group consisting of: a toxin, a radioisotope, an RNA polymerase II inhibitor and/or RNA polymerase III inhibitor, and a DNA-damaging agent.

In particular embodiments, the first antibody is conjugated to a cytotoxic agent that comprises a toxin.

Illustrative examples of toxins suitable for conjugation to a first antibody contemplated in particular embodiments include but are not limited to saporin, diphtheria toxin, pseudomonas exotoxin A, Ricin A chain derivatives, a small molecule toxin, and combinations thereof.

In particular embodiments, the first antibody is conjugated to a cytotoxic agent that comprises a radioisotope.

Illustrative examples of radioisotopes suitable for conjugation to a first antibody contemplated in particular embodiments include but are not limited to 131I, 90Y, 177Lu, 188Re, 67Cu, 213Bi, 211At, and 227Ac.

In particular embodiments, the first antibody is conjugated to a cytotoxic agent that comprises an RNA polymerase II and/or III inhibitor.

Illustrative examples of RNA polymerase II and/or III inhibitors suitable for conjugation to a first antibody contemplated in particular embodiments include but are not limited to an amatoxin. Suitable amatoxins include, but are not limited to α-amanitin, β-amanitin, γ-amanitin, ε-amanitin, amanin, amaninamide, amanullin, amanullinic acid and any functional fragments, derivatives or analogs thereof.

In particular embodiments, the first antibody is conjugated to a cytotoxic agent that comprises a DNA-damaging agent.

Illustrative examples of DNA-damaging agents suitable for conjugation to a first antibody contemplated in particular embodiments include but are not limited to an anti-tubulin agent, a DNA crosslinking agent, a DNA alkylating agent and a mitotic disrupting agent.

2. Second Antibody

In various embodiments, methods contemplated herein comprise administration of a second antibody that binds to a protein expressed on a hematopoietic stem cell or a protein expressed on a macrophage. Without wishing to be bound by any particular theory, it is contemplated that a class of proteins expressed on normal healthy hematopoietic stem and progenitor cells are able to associate with proteins expressed on macrophages and prevent engulfment of the normal healthy hematopoietic stem and progenitor cells (a “don't eat me” signal). It is further contemplated that when hematopoietic stem and/or progenitor cells are bound by a first antibody in the presence of a second antibody that blocks the “don't eat me signal”, macrophages can engulfment the hematopoietic stem and/or progenitor cells. Efficient removal of resident HSCs in the bone marrow makes room for donor HSCs to engraft.

In particular embodiments, the second antibody binds an antigen on a hematopoietic stem cell to enable macrophage engulfment of HSCs bound by the first antibody. In particular embodiments, the second antibody binds CD47.

In particular embodiments, the second antibody binds an antigen on a macrophage to enable macrophage engulfment of HSCs bound by the first antibody. In particular embodiments, the second antibody binds SIRPα.

Without wishing to be bound by any particular theory, it is contemplated that CD47 expressed on HSCs associates with SIRPα expressed on macrophages to provide don't eat me signal and prevent macrophage engulfment of HSCs. It is further contemplated that antibodies to CD47 or SIRPα block the CD47-SIRPα interaction and enable phagocytosis of HSCs macrophages, thereby making space in the subject's bone marrow for donor hematopoietic stem cells to engraft.

3. Antibody Endopeptidases

Methods contemplated herein comprise administration of an antibody-based RTC regimen to remove resident HSCs from the bone marrow to make space for donor HSCs to engraft. Without wishing to be by any particular theory, it is contemplated that antibody-based RTC conditioning has the caveat of requiring clearance of the antibodies prior to hematopoietic stem cell transplant because the RTC antibodies recognize proteins on the donor HSCs. Transplant may only be performed once RTC antibody clearance is demonstrated thereby increasing needle-to-needle times, extending the time that the transplant recipient in an immunocompromised state, and potentially interfering with and/or reducing the probability of successful HSC engraftment.

In particular embodiments, once the antibody-based RTC regimen is complete, the subject is administered an endopeptidase that degrades or digests and/or inhibits or reduces antibody effector function. In particular embodiments, the endopeptidase is a cysteine protease or a thiol protease.

Endopeptidases that degrade human antibodies have been identified in bacteria.

In particular illustrative embodiments, an endopeptidase that is suitable for use in degrading and/or clearing antibodies used in RTC regiments include but are not limited to endopeptidases isolated from Streptococcus pyogenes, Streptococcus equi, Streptococcus agalactiae, Streptococcus pseudoporcinus, Streptococcus suis, Streptococcus porcinus, Mycoplasma canis.

In particular embodiments, the endopeptidase is a protease comprising an IgG degrading enzyme (IgdE).

In preferred embodiments, the protease comprises an IgdE selected from the group consisting of: Immunoglobulin G-degrading enzyme of S. pyogenes (IdeS), Immunoglobulin G-degrading enzyme of S. equi ssp. zooepidemicus (IdeZ), an Immunoglobulin G-degrading enzyme of M. canis (IdeMC), and variants thereof.

In more preferred embodiments, the protease comprises an IdeS enzyme.

4. HSC Mobilization Agents

In particular methods contemplated herein, conditioning the subject comprises a step of hematopoietic stem cell mobilization before administration of a first antibody that binds to an antigen expressed on an HSC optionally conjugated to a cytotoxic agent, and optionally, administration of a second antibody that binds to another antigen expressed on an HSC or an antigen expressed on a macrophage, wherein the second antibody enables macrophages to recognize and engulf the HSCs bound by the first antibody. Without wishing to be bound by any particular theory, in particular embodiments, it is contemplated that mobilizing the HSCs prior to treatment with the first and optionally second antibodies enables the antibodies to more efficiently bind the antigens expressed on the HSCs, thereby enabling more efficient conditioning, and thereby increasing the efficiency of the HSC transplant or HSC-based therapy.

In particular methods contemplated herein, conditioning the subject comprises a step of hematopoietic stem cell mobilization in the absence of RTC antibodies.

In various embodiments, the subject is administered one or more mobilization agents selected from the group consisting of: GM-CSF, G-CSF (filgrastim), pegylated G-CSF (pegfilgrastim), glycosylated G-CSF (lenograstim), AMD3100 (plerixafor), macrophage inflammatory protein 1α (MIP1α), CCL3, SDF-1α peptide analogs (CTCE-0021, CTCE-0214), Met-SDF-1β, IL-8, GRO proteins (GRO-β (CXCL2)), SB-251353, and suitable combinations thereof. See e.g., Bakanay and Demirer, 2012. Bone Marrow Transplantation 47:1154-1163.

In particular embodiments, the subject is administered plerixafor and G-CSF or an analog thereof to mobilize the subject's HSCs.

In particular embodiments, the subject is administered plerixafor to mobilize the subject's HSCs.

In particular embodiments, a method of HSCT or an HSC-based gene therapy comprises administering the subject a first antibody, wherein the first antibody binds an antigen expressed on hematopoietic stem cells; and optionally administering the subject, a second antibody, wherein the second antibody blocks CD47 binding to SIRPα; administering the subject, an endopeptidase to degrade or digest and/or inhibit or reduce effector function of the first and second antibodies; and administering the subject, donor hematopoietic stem cells. In particular embodiments, the first antibody and the second antibody are each administered in a dose effective to ablate the hematopoietic stem cells in the subject's bone marrow. In particular embodiments, the first antibody and the second antibody are each administered in a dose effective to decrease the number of hematopoietic stem cells in the subject's bone marrow. In certain embodiments, the donor HSCs are CD34⁺. In certain embodiments, the donor HSCs are genetically modified. In certain embodiments, the donor HSCs comprise an HSC-based gene therapy. In particular embodiments, the subject is treated with one or more HSC mobilizing agents before being treated with a first and/or second antibody.

In particular embodiments, a method of HSCT or an HSC-based gene therapy comprises administering the subject a first antibody that binds c-kit (CD117), CD133, CD34, CD50, CD33, CD45, CD123, CD110, or CD90, optionally conjugated to a cytotoxic agent; and optionally administering the subject, a second antibody, wherein the second antibody is an anti-CD47 antibody or an anti-SIRPα antibody; administering the subject, an IgG degrading enzyme (IgdE) to degrade or digest and/or inhibit or reduce effector function of the first and second antibodies; and administering the subject, donor hematopoietic stem cells. In particular embodiments, the first antibody and the second antibody are each administered in a dose effective to ablate the hematopoietic stem cells in the subject's bone marrow. In particular embodiments, the first antibody and the second antibody are each administered in a dose effective to decrease the number of hematopoietic stem cells in the subject's bone marrow. In certain embodiments, the donor HSCs are CD34⁺. In certain embodiments, the donor HSCs are genetically modified. In certain embodiments, the donor HSCs comprise an HSC-based gene therapy. In particular embodiments, the subject is treated with one or more HSC mobilizing agents before being treated with a first and/or second antibody.

In particular embodiments, a method of HSCT or an HSC-based gene therapy comprises administering the subject an anti-c-kit antibody (first antibody), optionally conjugated to a cytotoxic agent; and optionally administering the subject, an anti-CD47 antibody or anti-SIRPα antibody that blocks CD47 binding to SIRPα (second antibody); administering the subject, an IgdE enzyme selected from the group consisting of: Immunoglobulin G-degrading enzyme of S. pyogenes (IdeS), Immunoglobulin G-degrading enzyme of S. equi ssp. zooepidemicus (IdeZ), an Immunoglobulin G-degrading enzyme of M. canis (IdeMC) and variants thereof to degrade or digest and/or inhibit or reduce effector function of the first and second antibodies; and administering the subject, donor hematopoietic stem cells. In particular embodiments, the first antibody and the second antibody are each administered in a dose effective to ablate the hematopoietic stem cells in the subject's bone marrow. In particular embodiments, the first antibody and the second antibody are each administered in a dose effective to decrease the number of hematopoietic stem cells in the subject's bone marrow. In certain embodiments, the donor HSCs are CD34⁺. In certain embodiments, the donor HSCs are genetically modified. In certain embodiments, the donor HSCs comprise an HSC-based gene therapy. In particular embodiments, the subject is treated with one or more HSC mobilizing agents before being treated with a first and/or second antibody.

In particular embodiments, a method of HSCT or an HSC-based gene therapy comprises administering the subject an anti-c-kit antibody (first antibody), optionally conjugated to a cytotoxic agent; and optionally administering the subject, an anti-CD47 antibody that blocks CD47 binding to SIRPα (second antibody); administering the subject, an IdeS enzyme or a variant thereof to degrade or digest and/or inhibit or reduce effector function of the first and second antibodies; and administering the subject, donor hematopoietic stem cells. In particular embodiments, the first antibody and the second antibody are each administered in a dose effective to ablate the hematopoietic stem cells in the subject's bone marrow. In particular embodiments, the first antibody and the second antibody are each administered in a dose effective to decrease the number of hematopoietic stem cells in the subject's bone marrow. In certain embodiments, the donor HSCs are CD34⁺. In certain embodiments, the donor HSCs are genetically modified. In certain embodiments, the donor HSCs comprise an HSC-based gene therapy. In particular embodiments, the subject is treated with one or more HSC mobilizing agents selected from the group consisting of: G-CSF and plerixafor, and plerixafor, before being treated with a first and/or second antibody.

The dosage and timing for administration of the one or more mobilizing agents to the subject may be performed according to established protocols. An effective dose of the one or more mobilizing agents can be administered for a duration suitable to mobilize HSCs from the subject's bone marrow. In particular embodiments, the subject is administered an effective dose of the one or more mobilizing agents for about 1 to about 6 days, or for about 1, about 2, about 3, about 4, about 5, about 6, about 7, about 8, about 9, or about 10 days, preferably for about 1, about 2, about 3, about 4, about 5 or about 6 days. In particular embodiments, the subject is administered an effective dose of the one or more mobilizing agents at least 1, at least 2, at least 3, at least 4, at least 5, at least 6, at least 7, at least 8, at least 9, or at least 10 days, preferably for at least 1, at least 2, at least 3, at least 4, at least 5 or at least 6 days. In particular embodiments, the subject is administered an effective dose of the one or more mobilizing agents for at most 1, at most 2, at most 3, at most 4, at most 5, at most 6, at most 7, at most 8, at most 9, or at most 10 days, preferably for at most 1, at most 2, at most 3, at most 4, at most 5 or at most 6 days.

In particular embodiments, in a conditioning method comprising a mobilization step, the timing for administration of the first antibody and the second antibody is preferably following the administration of the one or more mobilization agents. In particular embodiments, the first antibody, and optionally the second antibody are administered about 1 day to about 10 days or about 1 day to about 5 days after the subject has received the first or last dose of the one or more mobilization agents. In particular embodiments, the first antibody, and optionally the second antibody are administered about 1, about 2, about 3, about 4, about 5, about 6, about 7, about 8, about 9, or about 10 days after the subject has received the first or last dose of the one or more mobilization agents. In particular embodiments, the first antibody, and optionally the second antibody are administered at least 1, at least 2, at least 3, at least 4, at least 5, at least 6, at least 7, at least 8, at least 9, or at least 10 days after the subject has received the first or last dose of the one or more mobilization agents. In particular embodiments, the first antibody, and optionally the second antibody are administered at most 1, at most 2, at most 3, at most 4, at most 5, at most 6, at most 7, at most 8, at most 9, or at most 10 days after the subject has received the first or last dose of the one or more mobilization agents.

In particular embodiments, in the presence or absence or mobilization, the subject is administered one or more doses of the first antibody and optionally, one or more doses of the second antibody. In some embodiments, the first antibody and the second antibody are administered to the subject at about the same time. In some embodiments, the first antibody is administered to the subject before the second antibody is administered to the subject. In some embodiments, the first antibody is administered to the subject after the second antibody is administered to the subject.

The timing of administration of the endopeptidase that degrades or inactivates the first and the second antibodies is preferably about 1 day to about 14 days after the first or last dose of the first and/or second antibodies to the subject. In particular embodiments, the endopeptidase is administered to the subject about 1, about 2, about 3, about 4, about 5, about 6, about 7, about 8, about 9, about 10, about 11, about 12, about 13, or about 14 days or more after the first or last dose of the first and/or second antibodies to the subject. In particular embodiments, the endopeptidase is administered to the subject at least 1, at least 2, at least 3, at least 4, at least 5, at least 6, at least 7, at least 8, at least 9, at least 10, at least 11, at least 12, at least 13, or at least 14 days or more after the first or last dose of the first and/or second antibodies to the subject. In particular embodiments, the endopeptidase is administered to the subject at most 1, at most 2, at most 3, at most 4, at most 5, at most 6, at most 7, at most 8, at most 9, at most 10, at most 11, at most 12, at most 13, or at most 14 days or more after the first or last dose of the first and/or second antibodies to the subject. In particular embodiments, the endopeptidase will be administered to the subject after the first and optionally second antibodies have sufficiently removed or ablated the HSCs in the subject's bone marrow.

In particular embodiments, the subject is administered a population of donor cells comprising HSCs, optionally genetically modified HSCs, or an HSC-based gene therapy. In particular embodiments, the timing of administration of the donor HSCs, optionally genetically modified HSCs, or an HSC-based gene therapy is about 1 day to about 21 days, about 1 day to about 14 days, about 1 day to about 7 days, about 7 days to about 21 days, about 7 days to about 14 days, or about 1, about 2, about 2, about 4, about 5, about 6, about 7, about 8, about 9, about 10, about 11, about 12, about 13, about 14, about 15, about 16, about 17, about 18, about 19, about 20, or about 21 days after administration of the endopeptidase to the subject. In particular embodiments, the timing of administration of the donor HSCs, optionally genetically modified HSCs, or an HSC-based gene therapy is at least 1, at least 2, at least 2, at least 4, at least 5, at least 6, at least 7, at least 8, at least 9, at least 10, at least 11, at least 12, at least 13, at least 14, at least 15, at least 16, at least 17, at least 18, at least 19, at least 20, or at least 21 days after administration of the endopeptidase to the subject. In particular embodiments, the timing of administration of the donor HSCs, optionally genetically modified HSCs, or an HSC-based gene therapy is at most 1, at most 2, at most 2, at most 4, at most 5, at most 6, at most 7, at most 8, at most 9, at most 10, at most 11, at most 12, at most 13, at most 14, at most 15, at most 16, at most 17, at most 18, at most 19, at most 20, or at most 21 days after administration of the endopeptidase to the subject.

5. Chemotherapeutic Agents

In particular methods contemplated herein, conditioning the subject comprises administering an amount or dose of a chemotherapeutic agent effective to increase macrophage activation and recruitment to the bone marrow before administration of a first antibody that binds to an antigen expressed on an HSC optionally conjugated to a cytotoxic agent, and optionally, administration of a second antibody that binds to another antigen expressed on an HSC or an antigen expressed on a macrophage, wherein the second antibody enables macrophages to recognize and engulf the HSCs bound by the first antibody. Without wishing to be bound by any particular theory, in particular embodiments, it is contemplated that administration of a low or sub-ablative dose of a chemotherapeutic agent, an effective amount or dose, to the subject increases macrophage activation and recruitment to the bone marrow or hematopoietic stem cell niche and will improve clearance of the stem cell niche of resident HSCs and improve engraftment of donor HSCs, and thereby increase the efficiency of the HSC transplant or HSC-based therapy.

Illustrative examples of agents that are suitable for increasing macrophage recruitment and/or activation in particular embodiments include, but are not limited to low dose or sub-ablative doses of chemotherapeutic agents selected from the group consisting of cyclophosphamide, busulphan, treosulphan, melphalan, thiotepa, carboplatin, carmustine, etoposide, cytosine arabinoside (AraC), and fludarabine.

In particular embodiments, the subject is administered cyclophosphamide in a dose effective to increases macrophage activation and recruitment to the bone marrow or hematopoietic stem cell niche.

In particular embodiments, a method of HSCT or an HSC-based gene therapy comprises administering the subject an amount or dose of a chemotherapeutic agent effective to increase macrophage activation and recruitment to the bone marrow, a first antibody, wherein the first antibody binds an antigen expressed on hematopoietic stem cells; and optionally administering the subject, a second antibody, wherein the second antibody blocks CD47 binding to SIRPα; administering the subject, an endopeptidase to degrade or digest and/or inhibit or reduce effector function of the first and second antibodies; and administering the subject, donor hematopoietic stem cells. In particular embodiments, the first antibody and the second antibody are each administered in a dose effective to ablate the hematopoietic stem cells in the subject's bone marrow. In particular embodiments, the first antibody and the second antibody are each administered in a dose effective to decrease the number of hematopoietic stem cells in the subject's bone marrow. In certain embodiments, the donor HSCs are CD34⁺. In certain embodiments, the donor HSCs are genetically modified. In certain embodiments, the donor HSCs comprise an HSC-based gene therapy.

In particular embodiments, a method of HSCT or an HSC-based gene therapy comprises administering the subject an amount or dose of a chemotherapeutic agent effective to increase macrophage activation and recruitment to the bone marrow, wherein the agent is selected from cyclophosphamide, busulphan, treosulphan, melphalan, thiotepa, carboplatin, carmustine, etoposide, cytosine arabinoside (AraC), fludarabine or combination thereof; a first antibody that binds c-kit (CD117), CD133, CD34, CD50, CD33, CD45, CD123, CD110, or CD90, optionally conjugated to a cytotoxic agent; and optionally administering the subject, a second antibody, wherein the second antibody is an anti-CD47 antibody or an anti-SIRPα antibody; administering the subject, an IgG degrading enzyme (IgdE) to degrade or digest and/or inhibit or reduce effector function of the first and second antibodies; and administering the subject, donor hematopoietic stem cells. In particular embodiments, the first antibody and the second antibody are each administered in a dose effective to ablate the hematopoietic stem cells in the subject's bone marrow. In particular embodiments, the first antibody and the second antibody are each administered in a dose effective to decrease the number of hematopoietic stem cells in the subject's bone marrow. In certain embodiments, the donor HSCs are CD34⁺. In certain embodiments, the donor HSCs are genetically modified. In certain embodiments, the donor HSCs comprise an HSC-based gene therapy.

In particular embodiments, a method of HSCT or an HSC-based gene therapy comprises administering the subject an amount or dose of a chemotherapeutic agent effective to increase macrophage activation and recruitment to the bone marrow, wherein the agent is selected from cyclophosphamide, busulphan, treosulphan, melphalan, thiotepa, carboplatin, carmustine, etoposide, cytosine arabinoside (AraC), fludarabine or combination thereof; an anti-c-kit antibody (first antibody), optionally conjugated to a cytotoxic agent; and optionally administering the subject, an anti-CD47 antibody or anti-SIRPα antibody that blocks CD47 binding to SIRPα (second antibody); administering the subject, an IgdE enzyme selected from the group consisting of: Immunoglobulin G-degrading enzyme of S. pyogenes (IdeS), Immunoglobulin G-degrading enzyme of S. equi ssp. zooepidemicus (IdeZ), an Immunoglobulin G-degrading enzyme of M. canis (IdeMC) and variants thereof to degrade or digest and/or inhibit or reduce effector function of the first and second antibodies; and administering the subject, donor hematopoietic stem cells. In particular embodiments, the first antibody and the second antibody are each administered in a dose effective to ablate the hematopoietic stem cells in the subject's bone marrow. In particular embodiments, the first antibody and the second antibody are each administered in a dose effective to decrease the number of hematopoietic stem cells in the subject's bone marrow. In certain embodiments, the donor HSCs are CD34⁺. In certain embodiments, the donor HSCs are genetically modified. In certain embodiments, the donor HSCs comprise an HSC-based gene therapy.

In particular embodiments, a method of HSCT or an HSC-based gene therapy comprises administering the subject an amount or dose of a chemotherapeutic agent effective to increase macrophage activation and recruitment to the bone marrow, wherein the agent is selected from cyclophosphamide, busulphan, treosulphan, melphalan, thiotepa, carboplatin, carmustine, etoposide, cytosine arabinoside (AraC), fludarabine or combination thereof; an anti-c-kit antibody (first antibody), optionally conjugated to a cytotoxic agent; and optionally administering the subject, an anti-CD47 antibody that blocks CD47 binding to SIRPα (second antibody); administering the subject, an IdeS enzyme or a variant thereof to degrade or digest and/or inhibit or reduce effector function of the first and second antibodies; and administering the subject, donor hematopoietic stem cells. In particular embodiments, the first antibody and the second antibody are each administered in a dose effective to ablate the hematopoietic stem cells in the subject's bone marrow. In particular embodiments, the first antibody and the second antibody are each administered in a dose effective to decrease the number of hematopoietic stem cells in the subject's bone marrow. In certain embodiments, the donor HSCs are CD34⁺. In certain embodiments, the donor HSCs are genetically modified. In certain embodiments, the donor HSCs comprise an HSC-based gene therapy.

The dosage and timing for administration of the one or more chemotherapeutic agents to the subject may be performed according to established protocols. An effective dose of the one or more chemotherapeutic agents can be administered for a duration suitable to increase macrophage activation and migration or recruitment to the subject's bone marrow.

6. Donor Hematopoietic Stem Cells

The methods contemplated in particular embodiments herein comprise an antibody based reduced toxicity conditioning (RTC) regimen in combination with administration of an endopeptidase to clear, remove, and/or inactivate the RTC antibodies, followed by administration of a population of donor cells comprising hematopoietic stem cells (HSCs) to a subject. In particular embodiments, the donor cells comprise CD34⁺ hematopoietic stem cells. In particular embodiments, the donor cells comprise CD34⁺ selected hematopoietic stem cells.

In particular embodiments, the donor HSCs are autologous to the subject.

In particular embodiments, the donor HSCs are allogeneic to the subject.

In particular embodiments, the donor HSCs are xenogeneic to the subject.

In particular embodiments, the donor HSCs are not modified prior to administration to a subject. In particular embodiments, the donor HSCs are not genetically modified prior to administration to a subject.

In particular embodiments, the donor HSCs are modified prior to administration to a subject. In particular embodiments, the donor HSCs are genetically modified prior to administration to a subject. In certain embodiments, donor HSCs are genetically modified in vitro or ex vivo. Illustrative examples of genetic modification in particular embodiments include but are not limited to insertion, deletion, substitution, or replacement of one or more bases or base pairs in a cellular genome.

In particular embodiments, genetic modification comprises genome editing. In particular embodiments, genome editing may be carried out by introduction of one or more endonucleases including, by not limited to engineered homing endonucleases, megaTALs, TALENs, zinc finger nucleases, or CRISPR/CAS-based nuclease systems, e.g., CRISPR/CAS12a, CRISPR/CAS9. In particular embodiments, genome editing comprises a donor repair template comprising a polynucleotide sequence that will be inserted into the genome via homologous recombination. In particular embodiments, genome editing comprises editing in the absence of a donor repair template to disrupt a target sequence by non-homologous end-joining.

In particular embodiments, the donor HSCs are modified to generate an HSC-based gene therapy. In preferred embodiments, donor HSCs are modified by introducing a vector into the cell, the vector comprising a polynucleotide encoding a therapeutic transgene, including but not limited to a therapeutic polynucleotide, e.g., a single-stranded or double stranded inhibitory RNA, or therapeutic polypeptide. In particular embodiments, the vector is a non-viral vector. In particular embodiments, the vector is a viral vector. In particular embodiments, the vector is selected from the group consisting of an RNA, a plasmid, a transposon, a herpes simplex virus (HSV), an adenovirus (Ad), an adeno-associated virus (AAV), a retrovirus, and a lentivirus.

In various embodiments, a subject is administered an RTC antibody-based conditioning regimen, an endopeptidase, and an HSC transplant, wherein the HSCs have been modified with a vector that expresses a therapeutic transgene that provides curative, preventative, or ameliorative benefits to a subject diagnosed with or that is suspected of having a disease, disorder, or condition or a disease, disorder, or condition that is amenable to hematopoietic stem cell-based gene therapy.

Illustrative examples of disorders suitable for treatment with particular methods contemplated herein include, but are not limited to: autoimmune diseases, hemoglobinopathies, leukodystrophies, lysosomal storage diseases, and neurogenerative diseases.

Illustrative examples of disorders and corresponding therapeutic gene(s) that are suitable for use in particular methods contemplated herein include, but are not limited to: ADA-SCID (adenosine deaminase); Artemis protein deficiency (DCLRE1C); Batten disease (CLN1, CLN2, CLN3, CLN4, CLN5, CLN6, CLN7, CLN8, CLN10); β-thalassemia (β-globin, γ-globin, anti-sickling β-globin); sickle cell disease (β-globin, γ-globin, anti-sickling β-globin); cerebral ALD (ABCD1); chronic granulomatous diseases (cytochrome b-245 alpha chain, cytochrome b-245 beta chain, neutrophil cytosolic factor 1, neutrophil cytosolic factor 2, neutrophil cytosolic factor 4); Fabry disease (alpha-galactosidase A); Fanconi anemia (FANCA, FANCC, FANCG); Gaucher disease (glucocerebrosidase); Hemophilia A (Factor VIII); Hemophilia B (Factor IX); Leukocyte adhesion deficiency (CD18); metachromatic leukodystrophy (arylsulfatase A); MPS I (Hurler syndrome) (alpha-L-iduronidase); MPS II (Hunter's syndrome) (iduronate 2-sulfatase); MPS III (Sanfilippo Syndrome) (N-sulfoglucosamine sulfohydrolase); MPS IV-A (Morquio A Syndrome) (galactosamine-6 sulfatase); MPS IV-B (Morquio B Syndrome) (beta-galactosidase); MPS VI (Maroteaux-Lamy Syndrome) (N-acetylgalactosamine-4-sulphatase); Pompe Disease (acid alpha-glucosidase); RAG deficiency (RAG1/2); Wiskott Aldrich syndrome (WASP); X-linked lymphoproliferative syndrome (SH2D1A); and X-Linked Severe Combined Immunodeficiency (X-SCID) (IL2Rg).

In particular embodiments, a method comprises administering to a subject that has ADA-SCID a first antibody that binds to an antigen expressed on an HSC, e.g., c-kit (CD117), CD133, CD34, CD33, CD50, CD45, CD123, CD110 and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding adenosine deaminase.

In particular embodiments, a method comprises administering to a subject that has Artemis protein deficiency a first antibody that binds to an antigen expressed on an HSC, e.g., c-kit (CD117), CD133, CD34, CD33, CD50, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding DCLRE1C.

In particular embodiments, a method comprises administering to a subject that has Batten disease a first antibody that binds to an antigen expressed on an HSC, e.g., c-kit (CD117), CD133, CD34, CD33, CD50, CD45, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding CLN1, CLN2, CLN3, CLN4, CLN5, CLN6, CLN7, CLN8, or CLN10.

In particular embodiments, a method comprises administering to a subject that has a hemoglobinopathy a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding a globin protein.

In particular embodiments, the subject has β-thalassemia and is administered a cell-based gene therapy comprising hematopoietic stem cells comprising a vector that comprise a polynucleotide encoding a β-globin protein, a δ-globin protein, a γ-globin protein, an anti-sickling globin, a βA-T87Q-globin protein, a βA-G16D/E22A/T87Q-globin protein, or a βA-T87Q/K95E/K120E-globin protein.

In particular embodiments, the subject sickle cell disease and is administered a cell-based gene therapy comprising hematopoietic stem cells comprising a vector that comprise a polynucleotide encoding a β-globin protein, a δ-globin protein, a γ-globin protein, an anti-sickling globin, a βA-T87Q-globin protein, a βA-G16D/E22A/T87Q-globin protein, or a βA-T87Q/K95E/K120E-globin protein.

In particular embodiments, a method comprises administering to a subject that has a cerebral ALD a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding ABCD1.

In particular embodiments, a method comprises administering to a subject that has a chronic granulomatous disease a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding cytochrome b-245 alpha chain, cytochrome b-245 beta chain, neutrophil cytosolic factor 1, neutrophil cytosolic factor 2, or neutrophil cytosolic factor 4.

In particular embodiments, a method comprises administering to a subject that has Fabry disease a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding alpha-galactosidase A.

In particular embodiments, a method comprises administering to a subject that has Fanconi anemia a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding FANCA, FANCC, or FANCG.

In particular embodiments, a method comprises administering to a subject that has Gaucher disease a first antibody that binds to an antigen expressed on an HSC, e.g., c-kit (CD117), CD133, CD34, CD33, CD50, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding glucocerebrosidase.

In particular embodiments, a method comprises administering to a subject that has Hemophilia A a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding Factor VIII.

In particular embodiments, a method comprises administering to a subject that has Hemophilia B a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding Factor IX.

In particular embodiments, a method comprises administering to a subject that has Leukocyte adhesion deficiency a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding CD18.

In particular embodiments, a method comprises administering to a subject that has metachromatic leukodystrophy a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding arylsulfatase A.

In particular embodiments, a method comprises administering to a subject that has MPS I a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding alpha-L-iduronidase.

In particular embodiments, a method comprises administering to a subject that has MPS II a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding iduronate 2-sulfatase.

In particular embodiments, a method comprises administering to a subject that has MPS III a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding N-sulfoglucosamine sulfohydrolase.

In particular embodiments, a method comprises administering to a subject that has MPS IV-A a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding galactosamine-6 sulfatase.

In particular embodiments, a method comprises administering to a subject that has MPS IV-B a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding beta-galactosidase.

In particular embodiments, a method comprises administering to a subject that has MPS VI a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding N-acetylgalactosamine-4-sulphatase.

In particular embodiments, a method comprises administering to a subject that has Pompe Disease a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding acid alpha-glucosidase.

In particular embodiments, a method comprises administering to a subject that has RAG deficiency a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding RAG1/2.

In particular embodiments, a method comprises administering to a subject that has Wiskott Aldrich syndrome a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding WASP.

In particular embodiments, a method comprises administering to a subject that has X-linked lymphoproliferative syndrome a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding SH2D1A.

In particular embodiments, a method comprises administering to a subject that has—Linked Severe Combined Immunodeficiency a first antibody that binds to an antigen expressed on an HSC, e.g., c-kit (CD117), CD133, CD34, CD50, CD33, CD45, CD123, CD110, and CD90, optionally conjugated to a cytotoxic agent, and optionally, a second antibody that binds to another antigen expressed on an HSC, e.g., CD47, or an antigen expressed on a macrophage, e.g, SIRPα, wherein the second antibody enables macrophages to recognize and engulf the HSCs bound by the first antibody; administering an endopeptidase, e.g., IdeS or a variant thereof, that degrades or digests and/or inhibits or reduces effector function of the first antibody and the second antibody; and administering to the subject a cell-based gene therapy comprising hematopoietic stem cells comprising a vector that comprise a polynucleotide encoding IL2Rg.

In particular embodiments, mobilizing agents, antibodies, HSCs and other compositions are administered to a subject parenterally, preferably intravenously, e.g., infused intravenously.

In one illustrative embodiment, an effective amount of HSCs or an HSC-based gene therapy administered to a subject is at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, or at least 10×10⁶ cells/kg, or more cells/kg, including all intervening doses of cells.

In one illustrative embodiment, an effective amount of HSCs or an HSC-based gene therapy administered to a subject is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, or about 10×10⁶ cells/kg, or more cells/kg, including all intervening doses of cells.

In one illustrative embodiment, an effective amount of HSCs or an HSC-based gene therapy administered to a subject is from about 2×10⁶ cells/kg to about 10×10⁶ cells/kg, about 3×10⁶ cells/kg to about 10×10⁶ cells/kg, about 4×10⁶ cells/kg to about 10×10⁶ cells/kg, about 5×10⁶ cells/kg to about 10×10⁶ cells/kg, 2×10⁶ cells/kg to about 6×10⁶ cells/kg, 2×10⁶ cells/kg to about 7×10⁶ cells/kg, 2×10⁶ cells/kg to about 8×10⁶ cells/kg, 3×10⁶ cells/kg to about 6×10⁶ cells/kg, 3×10⁶ cells/kg to about 7×10⁶ cells/kg, 3×10⁶ cells/kg to about 8×10⁶ cells/kg, 4×10⁶ cells/kg to about 6×10⁶ cells/kg, 4×10⁶ cells/kg to about 7×10⁶ cells/kg, 4×10⁶ cells/kg to about 8×10⁶ cells/kg, 5×10⁶ cells/kg to about 6×10⁶ cells/kg, 5×10⁶ cells/kg to about 7×10⁶ cells/kg, 5×10⁶ cells/kg to about 8×10⁶ cells/kg, or 6×10⁶ cells/kg to about 8×10⁶ cells/kg, including all intervening doses of cells.

Some variation in dosage may be necessary depending on the condition of the subject and/or the condition being treated. Medical personnel responsible for treatment of the subject will, in any event, determine the appropriate dose for the individual subject.

### D. Polypeptides

Various polypeptides are contemplated herein, including, but not limited to, RTC antibodies, endopeptidases, polypeptide-based mobilization agents, and therapeutic polypeptides. “Polypeptide,” “peptide” and “protein” are used interchangeably, unless specified to the contrary, and according to conventional meaning, i.e., as a sequence of amino acids. In one embodiment, a “polypeptide” includes fusion polypeptide and other variants. Polypeptides can be prepared using any of a variety of well-known recombinant and/or synthetic techniques. Polypeptides are not limited to a specific length, e.g., they may comprise a full-length protein sequence, a fragment of a full-length protein, or a fusion protein, and may include post-translational modifications of the polypeptide, for example, glycosylations, acetylations, phosphorylations and the like, as well as other modifications known in the art, both naturally occurring and non-naturally occurring. In particular preferred embodiments, fusion polypeptides, polypeptides, fragments and other variants thereof are prepared, obtained, or isolated from one or more human polypeptides.

In particular embodiments, a polypeptide is selected from the group consisting of: ADA-SCID (adenosine deaminase); Artemis protein deficiency (DCLRE1C); Batten disease (CLN1, CLN2, CLN3, CLN4, CLN5, CLN6, CLN7, CLN8, CLN10); β-thalassemia (β-globin, γ-globin, anti-sickling β-globin); sickle cell disease (β-globin, γ-globin, anti-sickling β-globin); cerebral ALD (ABCD1); chronic granulomatous diseases (cytochrome b-245 alpha chain, cytochrome b-245 beta chain, neutrophil cytosolic factor 1, neutrophil cytosolic factor 2, neutrophil cytosolic factor 4); Fabry disease (alpha-galactosidase A); Fanconi anemia (FANCA, FANCC, FANCG); Gaucher disease (glucocerebrosidase); Hemophilia A (Factor VIII); Hemophilia B (Factor IX); Leukocyte adhesion deficiency (CD18); metachromatic leukodystrophy (arylsulfatase A); MPS I (Hurler syndrome) (alpha-L-iduronidase); MPS II (Hunter's syndrome) (iduronate 2-sulfatase); MPS III (Sanfilippo Syndrome) (N-sulfoglucosamine sulfohydrolase); Mucopolysaccharidosis type 4A—Morquio A Syndrome (galactosamine-6 sulfatase); Mucopolysaccharidosis type 4B—Morquio B Syndrome (beta-galactosidase); Mucopolysaccharidosis type 6—Maroteaux-Lamy Syndrome (N-acetylgalactosamine-4-sulphatase); Pompe Disease (acid alpha-glucosidase); Wiskott Aldrich syndrome (WASP); X-linked lymphoproliferative syndrome (SH2D1A), RAG deficiency (RAG1/2); and X-Linked Severe Combined Immunodeficiency (X-SCID) (IL2Rg).

In particular embodiments, a polypeptide is an antibody or antigen binding fragment thereof that binds an antigen expressed on an HSC including, but not limited to, c-kit (CD117), CD133, CD34, CD50, CD33, CD45, CD123, CD110, and CD90.

In particular embodiments, a polypeptide is an antibody or antigen binding fragment thereof that binds an antigen expressed on a hematopoietic stem cell to enable macrophage engulfment of HSCs. In particular embodiments, a polypeptide is an anti-CD47 antibody or antigen binding fragment thereof or an anti-SIRPα antibody or antigen binding fragment thereof.

In particular embodiments, a polypeptide is an endopeptidase comprising an IgG degrading enzyme (IgdE). In preferred embodiments, the polypeptide is an endopeptidase selected from the group consisting of: Immunoglobulin G-degrading enzyme of S. pyogenes (IdeS), Immunoglobulin G-degrading enzyme of S. equi ssp. zooepidemicus (IdeZ), an Immunoglobulin G-degrading enzyme of M. canis (IdeMC), and variants thereof. In more preferred embodiments, polypeptide is an IdeS enzyme.

In various embodiments, a polypeptide is a mobilization agent selected from the group consisting of: GM-CSF, G-CSF (filgrastim), pegylated G-CSF (pegfilgrastim), glycosylated G-CSF (lenograstim), macrophage inflammatory protein 1α (MIP1α), CCL3, SDF-1α peptide analogs (CTCE-0021, CTCE-0214), Met-SDF-1β, IL-8, and GRO proteins (GRO-β (CXCL2)).

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. In particular embodiments, an isolated polypeptide is a synthetic polypeptide, a semi-synthetic polypeptide, or a polypeptide obtained or derived from a recombinant source.

Polypeptides include “polypeptide variants.” Polypeptide variants may differ from a naturally occurring polypeptide in one or more substitutions, deletions, additions and/or insertions. Such variants may be naturally occurring or may be synthetically generated, for example, by modifying one or more of the above polypeptide sequences. For example, in particular embodiments, it may be desirable to improve the binding affinity and/or other biological properties of a polypeptide by introducing one or more substitutions, deletions, additions and/or insertions the polypeptide. In particular embodiments, polypeptides include polypeptides having at least about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% amino acid identity to any of the reference sequences contemplated herein, typically where the variant maintains at least one biological activity of the reference sequence. In particular embodiments, the biological activity is binding affinity. In particular embodiments, the biological activity is enzymatic activity.

Polypeptides variants include biologically active “polypeptide fragments.” Illustrative examples of biologically active polypeptide fragments include binding domains, intracellular signaling domains, and the like. As used herein, the term “biologically active fragment” or “minimal biologically active fragment” refers to a polypeptide fragment that retains at least 100%, at least 90%, at least 80%, at least 70%, at least 60%, at least 50%, at least 40%, at least 30%, at least 20%, at least 10%, or at least 5% of the naturally occurring polypeptide activity. In certain embodiments, a polypeptide fragment can comprise an amino acid chain at least 5 to about 1700 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, 450, 500, 550, 600, 650, 700, 750, 800, 850, 900, 950, 1000, 1100, 1200, 1300, 1400, 1500, 1600, 1700 or more amino acids long.

In particular embodiments, the polypeptides set forth herein may comprise one or more amino acids denoted as “X.” “X” if present in an amino acid SEQ ID NO, refers to any one or more amino acids. In particular embodiments, SEQ ID NOs denoting a fusion protein comprise a sequence of continuous X residues that cumulatively represent any amino acid sequence.

As noted above, polypeptides may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

### E. Polynucleotides

In particular embodiments, polynucleotides encoding RTC antibodies, endopeptidases, polypeptide-based mobilization agents, and therapeutic polypeptides are provided. As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded and either recombinant, synthetic, or isolated. Polynucleotides include, but are not limited to: pre-messenger RNA (pre-mRNA), messenger RNA (mRNA), RNA, short interfering RNA (siRNA), short hairpin RNA (shRNA), microRNA (miRNA), ribozymes, genomic RNA (gRNA), plus strand RNA (RNA(+)), minus strand RNA (RNA(−)), tracrRNA, crRNA, single guide RNA (sgRNA), synthetic RNA, synthetic mRNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

As used herein, “isolated polynucleotide” refers to a polynucleotide that has been purified from the sequences which flank it in a naturally-occurring state, e.g., a DNA fragment that has been removed from the sequences that are normally adjacent to the fragment. An “isolated polynucleotide” also refers to a complementary DNA (cDNA), a recombinant DNA, or other polynucleotide that does not exist in nature and that has been made by the hand of man. In particular embodiments, an isolated polynucleotide is a synthetic polynucleotide, a semi-synthetic polynucleotide, or a polynucleotide obtained or derived from a recombinant source.

Illustrative examples of polynucleotides include, but are not limited to, polynucleotides encoding ADA-SCID (adenosine deaminase); Artemis protein deficiency (DCLRE1C); Batten disease (CLN1, CLN2, CLN3, CLN4, CLN5, CLN6, CLN7, CLN8, CLN10); β-thalassemia (β-globin, γ-globin, anti-sickling β-globin); sickle cell disease (β-globin, γ-globin, anti-sickling β-globin); cerebral ALD (ABCD1); chronic granulomatous diseases (cytochrome b-245 alpha chain, cytochrome b-245 beta chain, neutrophil cytosolic factor 1, neutrophil cytosolic factor 2, neutrophil cytosolic factor 4); Fabry disease (alpha-galactosidase A); Fanconi anemia (FANCA, FANCC, FANCG); Gaucher disease (glucocerebrosidase); Hemophilia A (Factor VIII); Hemophilia B (Factor IX); Leukocyte adhesion deficiency (CD18); metachromatic leukodystrophy (arylsulfatase A); MPS I (Hurler syndrome) (alpha-L-iduronidase); MPS II (Hunter's syndrome) (iduronate 2-sulfatase); MPS III (Sanfilippo Syndrome) (N-sulfoglucosamine sulfohydrolase); Mucopolysaccharidosis type 4A—Morquio A Syndrome (galactosamine-6 sulfatase); Mucopolysaccharidosis type 4B—Morquio B Syndrome (beta-galactosidase); Mucopolysaccharidosis type 6—Maroteaux-Lamy Syndrome (N-acetylgalactosamine-4-sulphatase); Pompe Disease (acid alpha-glucosidase); Wiskott Aldrich syndrome (WASP); X-linked lymphoproliferative syndrome (SH2D1A), RAG deficiency (RAG1/2); or X-Linked Severe Combined Immunodeficiency (X-SCID) (IL2Rg).

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion, substitution, or modification of at least one nucleotide. Accordingly, the terms “polynucleotide variant” and “variant” include polynucleotides in which one or more nucleotides have been added or deleted, or modified, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide.

The polynucleotides contemplated herein, regardless of the length of the coding sequence itself, may be combined with other DNA sequences, e.g., expression control sequences such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

“Expression control sequences,” “control elements,” or “regulatory sequences” present in an expression vector are those non-translated regions of the vector including an origin of replication, selection cassettes, promoters, enhancers, translation initiation signals (Shine Dalgarno sequence or Kozak sequence) introns, a polyadenylation sequence, 5′ and 3′ untranslated regions, all of which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, a polynucleotide comprises one or more exogenous, endogenous, or heterologous control sequences such as promoters and/or enhancers. An “endogenous control sequence” is one which is naturally linked with a given gene in the genome. An “exogenous control sequence” is one which is placed in juxtaposition to a gene by means of genetic manipulation (i.e., molecular biological techniques) such that transcription of that gene is directed by the linked enhancer/promoter. A “heterologous control sequence” is an exogenous sequence that is from a different species than the cell being genetically manipulated. A “synthetic” control sequence may comprise elements of one more endogenous and/or exogenous sequences, and/or sequences determined in vitro or in silico that provide optimal promoter and/or enhancer activity for the particular therapy.

The term “promoter” as used herein refers to a recognition site of a polynucleotide (DNA or RNA) to which an RNA polymerase binds. The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

Illustrative examples of expression control sequences suitable for use in particular embodiments include, but are not limited to: a cytomegalovirus (CMV) immediate early promoter, a viral simian virus 40 (SV40) (e.g., early or late) promoter, a Moloney murine leukemia virus (MoMLV) LTR promoter, a Rous sarcoma virus (RSV) LTR promoter, a herpes simplex virus (HSV) (thymidine kinase) promoter, H5, P7.5, and P11 promoters from vaccinia virus, an elongation factor 1-alpha (EF1a) promoter, a Glyceraldehyde 3-phosphate dehydrogenase (GAPDH) promoter, an eukaryotic translation initiation factor 4A1 (EIF4A1) promoter, a heat shock protein 70 kDa (HSP70) promoter, a human ROSA 26 locus promoter (Irions et al., (2007) Nature Biotechnology 25, 1477-1482), a Ubiquitin C promoter (UBC) promoter, a phosphoglycerate kinase-1 (PGK) promoter, a cytomegalovirus enhancer/chicken β-actin (CAG) promoter, and a β-actin promoter.

Additional illustrative examples of expression control sequences suitable for use in particular embodiments include, but are not limited to: hematopoietic cell or tissue specific promoters and/or enhancers selected from the group consisting of: a human β-globin promoter; a human β-globin LCR; and a human α-globin HS40 enhancer and an ankyrin-1 promoter, operably linked to a polynucleotide encoding a globin polypeptide.

Further illustrative examples of expression control sequences suitable for use in particular embodiments include, but are not limited to: a microglial cell promoters selected from the group consisting of: an integrin subunit alpha M (ITGAM; CD11b) promoter, a CD68 promoter, a C-X3-C motif chemokine receptor 1 (CX3CR1) promoter, an ionized calcium binding adaptor molecule 1 (IBA1) promoter, a transmembrane protein 119 (TMEM119) promoter, a spalt like transcription factor 1 (SALL1) promoter, an adhesion G protein-coupled receptor E1 (F4/80) promoter, a myeloproliferative sarcoma virus enhancer, negative control region deleted, dl587rev primer-binding site substituted U3 (MNDU3) promoter.

Polynucleotides can be prepared, manipulated, expressed and/or delivered using any of a variety of well-established techniques known and available in the art. In order to express a desired polypeptide, a nucleotide sequence encoding the polypeptide, can be inserted into appropriate vector.

Illustrative examples of vectors include, but are not limited to plasmid, autonomously replicating sequences, and transposable elements, e.g., Sleeping Beauty, PiggyBac.

Additional Illustrative examples of vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses.

Illustrative examples of expression vectors include, but are not limited to, pClneo vectors (Promega) for expression in mammalian cells; pLenti4/V5-DEST™ pLenti6/V5-DEST™, and pLenti6.2/V5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

Viral vectors comprising polynucleotides contemplated in particular embodiments can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, viral vectors comprising polynucleotides contemplated herein are administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Illustrative examples of viral vector systems suitable for use in particular embodiments contemplated in particular embodiments include, but are not limited to, adeno-associated virus (AAV), retrovirus, herpes simplex virus, adenovirus, and vaccinia virus vectors, among others.

In various embodiments, one or more polynucleotides are introduced into an HSC by transducing the cell with a recombinant adeno-associated virus (rAAV), comprising the one or more polynucleotides. Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety.

In various embodiments, one or more polynucleotides are introduced into an HSC by transducing the cell with a retrovirus, e.g., lentivirus, comprising the one or more polynucleotides. Illustrative examples of retroviral vectors are disclosed in Naldini et al., (1996a, 1996b, and 1998); Zufferey et al., (1997); Dull et al., 1998, U.S. Pat. Nos. 6,013,516; and 5,994,136, each of which is incorporated by reference herein, in its entirety. Lentiviral vectors can be produced according to known methods. See e.g., Kutner et al., BMC Biotechnol. 2009; 9:10. doi: 10.1186/1472-6750-9-10; Kutner et al. Nat. Protoc. 2009; 4(4):495-505. doi: 10.1038/nprot.2009.22.

In various embodiments, one or more polynucleotides are introduced into an HSC by transducing the cell with an adenovirus comprising the one or more polynucleotides. Adenovirus vectors have been used in eukaryotic gene expression (Levrero et al., 1991; Gomez-Foix et al., 1992; Rosenfeld et al., 1991; Rosenfeld et al., 1992; Ragot et al., 1993; Herz & Gerard, 1993; Le Gal La Salle et al., 1993; and Sterman et al., Hum. Gene Ther. 7:1083-9 (1998)), each of which is incorporated by reference herein, in its entirety.

In various embodiments, one or more polynucleotides are introduced into an HSC by transducing the cell with a herpes simplex virus, e.g., HSV-1, HSV-2, comprising the one or more polynucleotides. HSV-based vectors are described in, for example, U.S. Pat. Nos. 5,837,532, 5,846,782, and 5,804,413, and International Patent Applications WO 91/02788, WO 96/04394, WO 98/15637, and WO 99/06583, each of which are incorporated by reference herein in its entirety.

### F. Compositions and Formulations

The compositions contemplated herein may comprise one or more HSC mobilization agents, chemotherapeutic agents, RTC antibodies, antibody endopeptidases, polynucleotides, vectors comprising same, hematopoietic stem cells grafts and/or cell therapies comprising genetically modified hematopoietic stem cells. Compositions include, but are not limited to pharmaceutical compositions. A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, pro-drugs, drugs, antibodies, or other various pharmaceutically-active agents. There is virtually no limit to other components that may also be included in the compositions, provided that the additional agents do not adversely affect the ability of the composition to deliver the intended therapy.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular embodiments, compositions comprise an amount of an RTC conditioning antibody, an antibody endopeptidase, an HSC mobilization agent, or a population of cells comprising hematopoietic stem cells contemplated herein.

As used herein, the term “amount” refers to “an amount effective” or “an effective amount” of a mobilization agent, an RTC antibody, HSCs, an HSC-based gene therapy, or other composition contemplated herein effective to achieve a beneficial or desired prophylactic or therapeutic result, including clinical results.

A “prophylactically effective amount” refers to an amount of a mobilization agent, an RTC antibody, HSCs, an HSC-based gene therapy, or other composition contemplated herein effective to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a mobilization agent, an RTC antibody, HSCs, an HSC-based gene therapy, or other composition contemplated herein may vary according to factors such as the disease state, age, sex, and weight of the individual, and its ability to elicit a desired response in the individual. A therapeutically effective amount is one in which any toxic or detrimental effects are outweighed by the therapeutically beneficial effects. The term “therapeutically effective amount” includes an amount that is effective to “treat” a subject (e.g., a patient).

Generally, compositions comprising the cells contemplated herein may be utilized in the treatment and prevention of diseases that arise in individuals who would benefit from receiving a hematopoietic stem cell transplant or gene therapy.

In particular, compositions contemplated herein are used in the treatment of an autoimmune disease, a hemoglobinopathy, a leukodystrophy, a lysosomal storage disease, and a neurogenerative disease.

Illustrative examples of diseases, disorders, or conditions that can be treated with compositions and methods contemplated in particular embodiments herein include, but are not limited to, ADA-SCID, Artemis protein deficiency, Batten disease, β-thalassemia, sickle cell disease, cerebral ALD, chronic granulomatous diseases, Fabry disease, Fanconi anemia, Gaucher disease, Hemophilia A, Hemophilia B, Leukocyte adhesion deficiency, metachromatic leukodystrophy, MPS I (Hurler syndrome), MPS II (Hunter's syndrome), MPS III (Sanfilippo Syndrome), MPS IV-A (Morquio A Syndrome), MPS IV-B (Morquio B Syndrome), MPS VI (Maroteaux-Lamy Syndrome), Pompe Disease (acid alpha-glucosidase); RAG deficiency, Wiskott Aldrich syndrome, X-linked lymphoproliferative syndrome, and X-Linked Severe Combined Immunodeficiency (X-SCID).

In particular embodiments, pharmaceutical compositions comprise an amount of genetically modified cells, e.g., HSCs, in combination with one or more pharmaceutically or physiologically acceptable carriers, diluents or excipients.

Pharmaceutical compositions comprising a cell population, such as HSCs may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. In particular embodiments, compositions are preferably formulated for nasal, oral, enteral, or parenteral administration, e.g., intravascular (intravenous or intraarterial), intraperitoneal or intramuscular administration.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In one embodiment, the cell compositions contemplated herein are formulated in a pharmaceutically acceptable cell culture medium. Such compositions are suitable for administration to human subjects. In particular embodiments, the pharmaceutically acceptable cell culture medium is a serum free medium.

Serum-free medium has several advantages over serum containing medium, including a simplified and better defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions includes but is not limited to QBSF-60 (Quality Biological, Inc.), StemPro-34 (Life Technologies), and X-VIVO 10.

In one preferred embodiment, compositions comprising HSCs contemplated herein are formulated in a solution comprising PlasmaLyte A.

In another preferred embodiment, compositions comprising HSCs contemplated herein are formulated in a solution comprising a cryopreservation medium. For example, cryopreservation media with cryopreservation agents may be used to maintain a high cell viability outcome post-thaw. Illustrative examples of cryopreservation media used in particular compositions includes, but is not limited to, CryoStor CS10, CryoStor CS5, and CryoStor CS2.

In a more preferred embodiment, compositions comprising HSCs contemplated herein are formulated in a solution comprising 50:50 PlasmaLyte A to CryoStor CS10.

In a particular embodiment, compositions comprise an effective amount of HSCs, alone or in combination with one or more therapeutic agents.

In particular embodiments, formulation of pharmaceutically-acceptable carrier solutions is well-known to those of skill in the art, as is the development of suitable dosing and treatment regimens for using the particular compositions described herein in a variety of treatment regimens, including e.g., enteral and parenteral, e.g., intravascular, intravenous, intrarterial, intraosseously, intraventricular, intracerebral, intracranial, intraspinal, intrathecal, and intramedullary administration and formulation. It would be understood by the skilled artisan that particular embodiments contemplated herein may comprise other formulations, such as those that are well known in the pharmaceutical art, and are described, for example, in Remington: The Science and Practice of Pharmacy, volume I and volume II. 22^{nd} Edition. Edited by Loyd V. Allen Jr. Philadelphia, PA: Pharmaceutical Press; 2012, which is incorporated by reference herein, in its entirety.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### IdeZ Cleaves Reduced Toxicity Conditioning Antibodies In Vitro

The purpose of this experiment was to explore the use of an immunoglobulin G (IgG)-degrading enzyme from Streptococcus equi subspecies zooepidemicus (IdeZ) to cleave antibodies used for reduced toxicity conditioning. IdeZ was used because it has significantly improved activity against mouse IgG2a and IgG3 subclasses compared to immunoglobulin G (IgG)-degrading enzyme from Streptococcus pyogenes (IdeS). IdeZ protease does not cleave mouse IgG1 or IgG2b.

### In Vitro Evaluation

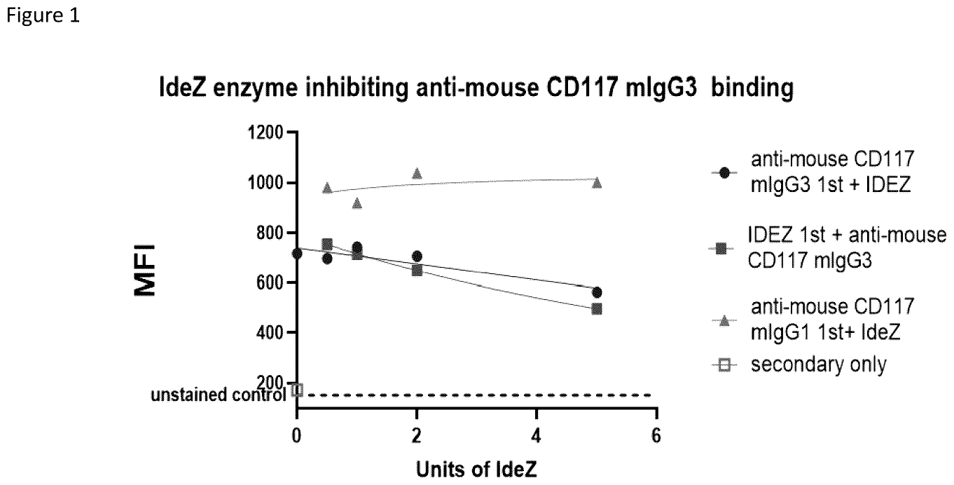

A murine basophil cell line expressing CD117 (EML cells) was incubated with various concentrations of anti-CD117 antibody (clone ACK2) or the matching murine IgG1 negative control. IdeZ (range of 0 to 5 units) was added before or after antibody addition to cells. Antibody was incubated with cells for 2 hours at 37° C. Cells will be washed with phosphate buffered saline (PBS) and subsequently incubated with anti-hIgG secondary antibody conjugated to a fluorophore. Cells were analyzed with flow cytometry and mean fluorescence intensity was used to measure binding affinity. Decreased binding affinity in the presence of IdeZ was observed in the samples with a murine IgG3, but not a murine IgG1 (negative control). FIG. 1.

### Example 2

### Reduced Toxicity Conditioning Using Ides

The purpose of this experiment is to explore the use of IdeS in a reduced toxicity conditioning regimen prior to hematopoietic stem cell transplant.

### In Vivo Evaluation

Immunocompetent mice (C57Bl/6 or Balb/C) will be dosed with anti-CD117 (clone ACK2) and anti-CD47 (clone MIAP410) antibodies to eliminate murine hematopoietic stem cells (HSC). Anti-CD47 antibody will be dosed at 1-50 mg/kg daily intraperitoneally for 4-10 days. Anti-CD117 antibody will be delivered intravascularly at a dose of 0.1-50 mg/kg once. Some mice will also receive IdeS at 0.1-10 mg/kg intraperitoneally for 1-5 days during or following treatment with the conditioning antibodies. Kinetics of antibody cleavage and elimination of the conditioning antibodies by IdeS will be measured in serum by ELISA with CD117 protein capture and anti-hIgG for detection.

Depletion of HSCs in the bone marrow of mice treated with IdeS will be compared to those treated without IdeS to ensure HSC depletion is comparable. Mice will be euthanized 5-21 days after final antibody or IdeS treatment and bone marrow will be harvested. Flow cytometry analysis will be performed to determine the proportion of depleted cells for each lineage. Briefly, bone marrow cells will be stained with an antibody cocktail containing the following antibodies: CD45, CD11b, CD3, CD19, Ter119, Lineage cocktail, Sca-1, c-KIT, CD48, CD150. Viability dyes will also be included in the analysis.

Upon confirmation of equivalent depletion levels between IdeS and non-IdeS treated groups, engraftment experiments will be performed. Briefly, fully immunocompetent C57Bl/6 CD45.1/CD45.2 adult mice will be treated with anti-CD117 and anti-CD47 antibodies with and without IdeS as described above. 0-28 days after treatment and/or IdeS dosing, mice will be injected with 0.5×10⁶-1.0×10⁶ lineage negative or whole bone marrow cells from C57Bl/6 CD45.2 animals. In some groups, lineage negative bone marrow cells may be transduced with a lentiviral vector with or without a transgene. Control groups will include animals not treated with IdeS as well as animals conditioned with irradiation (400-1000 cGy) or busulfan (4 doses of 25 mg/kg). IdeS dosing timing will be determined by depletion and antibody serum kinetics. A time course of transplant following antibody and/or IdeS dosing will be performed to demonstrate that IdeS cleavage of the antibody conditioning regimen allows for earlier yet equally efficient engraftment.

Engraftment efficiency will be measured by flow cytometry in the peripheral blood and bone marrow for the CD45 variants for 4-24 weeks after transplant. For transplanted cells modified by a lentiviral vector, relevant flow cytometric and molecular assays including qPCR for vector presence will be completed to determine the level of gene marking. Engraftment efficiency in the peripheral blood and bone marrow of mice treated with and without IdeS will be compared to ensure engraftment is not hindered with the use of IdeS.

### Example 3

### Cyclophosphamide Increases Activated Macrophage Recruitment and Anti-CD117 Mediated Stem Cell Depletion in Bone Marrow

Immunocompetent mice (C57Bl/6 or Balb/C) were dosed with vehicle of one of four different cyclophosphamide (Cytoxan) regimens including: one dose of 50 mg/kg, one dose of 100 mg/kg, two doses of 50 mg/kg approximately 48 hours apart, and two doses of 100 mg/kg approximately 48 hours apart. Three days following the final dose of the regimen, mouse bone marrow was evaluated for macrophage number and phenotype of. Flow cytometry was used to characterize bone marrow macrophages. Briefly, bone marrow was stained with murine macrophage markers including CD11b, F4/80, CD16 (FcγRIII; marker of activated macrophages), CD32 (FcγRII; inhibitory macrophage marker), CD68, and CD64 (FcγRI; marker of activated macrophages). In addition, bone marrow smears were prepared for pathology analysis and stained with F4/80 to quantify macrophages.

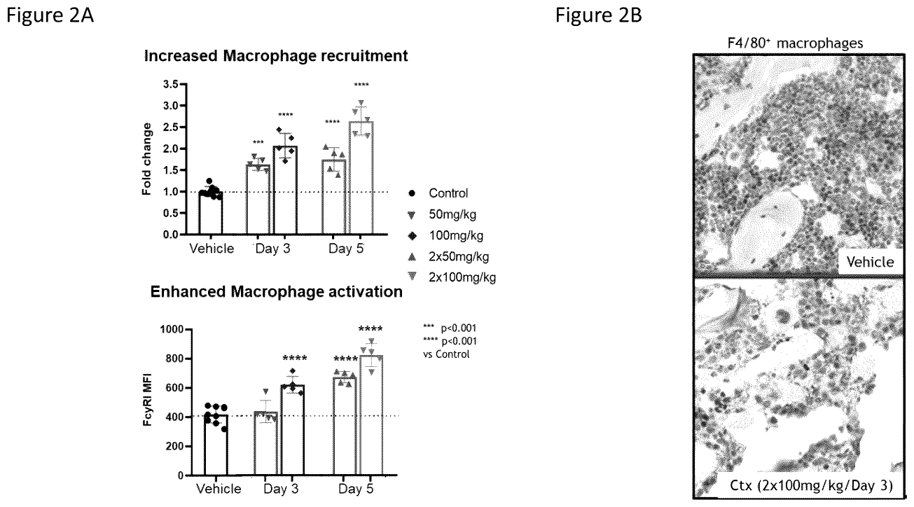

Cyclophosphamide increased macrophage content in the bone marrow up to 2.5-fold. FIG. 2A, top panel. In addition, macrophages recruited to the bone marrow showed enhanced activation status as evidenced by increased FcγRI signal. FIG. 2A, bottom panel. Pathology confirmed the increase in F4/80⁺ macrophages in the bone marrow. FIG. 2B.

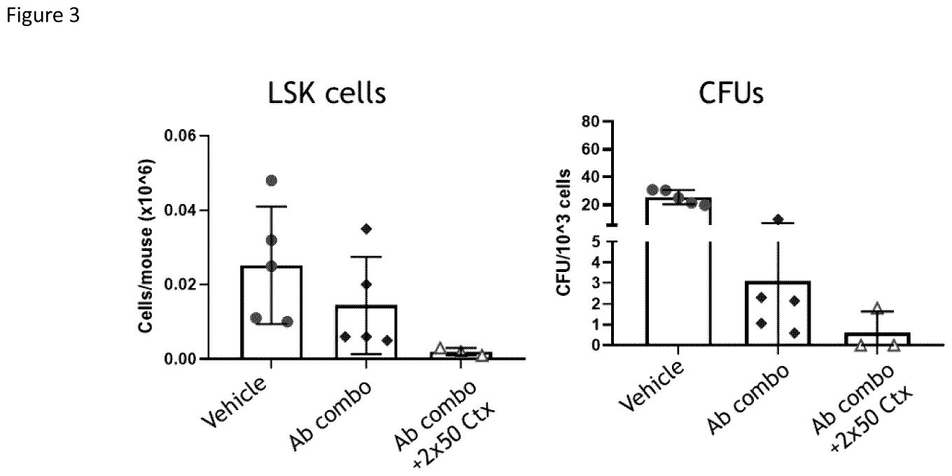

In another experiment, immunocompetent mice (C57Bl/6 or Balb/C) were dosed with 8.3-25 mg/kg of anti-CD117 and anti-CD47 antibodies with or without two doses of 50 mg/kg of cyclophosphamide approximately 48 hours apart. Five to seven days after antibody treatment, mouse bone marrow was analyzed by flow cytometry or plated in methocellulose medium to evaluate impacts on the hematopoietic stem cells. Treatment with cyclophosphamide enhanced the antibody-mediated depletion of stem cells (LSK: lineage negative, Sca1+, ckit+) and reduced the number of colony forming cells (CFUs) in the bone marrow. FIG. 3.

### Example 4

### Use of Mobilization Agents in Reduced Toxicity Conditioning

Immunocompetent mice (C57Bl/6 or Balb/C) will be dosed with anti-CD117 (clone ACK2) and anti-CD47 (clone MIAP410) antibodies to eliminate murine hematopoietic stem cells (HSC). Anti-CD47 antibody will be dosed at 1-50 mg/kg daily intraperitoneally for 4-10 days. Anti-CD117 antibody will be delivered intravascularly at a dose of 0.1-50 mg/kg once. Some mice will also receive mobilization agents including granulocyte colony stimulating factor [G-CSF] and/or Plerixafor [AMD3100]. Mice receiving mobilization agents will receive 250 ug/kg of G-CSF over four days and a single dose of Plerixafor at 5 mg/kg. Control groups will include vehicle (PBS), Busulfan-treated as an existing standard regimen, mobilization agents only, and antibodies only. To confirm mobilization, a peel-off group of animals will be euthanized following the four-day regimen of G-CSF and/or the one hour treatment with Plerixafor. Blood will be analyzed by flow cytometry and plated in methocellulose to quantify the number of hematopoietic stem cells in the peripheral blood of the mice compared to control animals. In addition, upon completion of the antibody conditioning regimen, a peel-off group of animals will be euthanized to evaluate the levels of hematopoietic stem cell depletion in the bone marrow by flow cytometry. Briefly, bone marrow cells will be stained with an antibody cocktail containing the following antibodies: CD45, CD11b, CD3, CD19, Ter119, Lineage cocktail, Sca-1, c-KIT, CD48, CD150. Viability dyes will also be included in the analysis.

### Engraftment

Upon confirmation of sufficient mobilization of HSCs into the peripheral blood and depletion of LSK cells, engraftment experiment will be performed. Following completion of the antibody and/or mobilization agent dosing, 0-28 days later, mice will be injected with 0.5-10e6 lineage negative or whole bone marrow cells from C57Bl/6 CD45.2 animals. In some groups, lineage negative bone marrow cells may be transduced with a lentiviral vector with or without a transgene. Control groups will include animals not treated with mobilization agents as well as animals receiving conditioning via irradiation (400-1000 cGy) or busulfan (4 doses of 25 mg/kg). A time course of delivery of mobilization agents and antibodies may be performed.

Engraftment efficiency will be measured by flow cytometry in the peripheral blood and bone marrow for the CD45 variants for 4-24 weeks after transplant. In the cases where the transplanted cells have been modified by a lentiviral vector, relevant flow cytometric and molecular assays including qPCR for vector presence will be completed to determine the level of gene marking in transplanted animals. Engraftment efficiency in the peripheral blood and bone marrow of mice treated with and without mobilization will be compared to determine whether mobilization increases the levels of engraftment.

### Non-Human Primate

Experiments similar to the ones above will be performed in a non-human primate model of autologous gene therapy. Briefly, rhesus macaques or other relevant non-human primates will be treated with anti-CD117 and anti-CD47 antibodies to eliminate hematopoietic stem cells (HSCs). Anti-CD47 antibody will be dosed at 1-50 mg/kg for 1-30 days. Anti-CD117 antibody will be delivered at a dose of 0.1-50 mg/kg once. The primates will also receive mobilization agents including granulocyte colony stimulating factor [G-CSF] and/or Plerixafor [AMD3100]. Following this conditioning regimen, animals will be infused with 5-10e6 cell/kg of gene-modified autologous cells (possibly marked with a lentiviral vector for tracking). Bone marrow and peripheral blood will be sampled from the animals at one, two, four, and eight weeks as well as three, six, nine, and twelve months to monitor engraftment of the gene modified cells. Engraftment efficiency will be determined based on the molecular presence of the vector (by qPCR) and normalized based on the percentage of marked cells in the graft at the time of infusion. Engraftment efficiency in animals receiving the antibody cocktail in addition to the mobilization agents will be compared to those receiving the antibody cocktail alone or historical controls conditioned with total body irradiation.

### Example 5

### Reduced Toxicity Conditioning with HSC-Targeting Antibodies

Immunocompetent mice (C57Bl/6 or Balb/C) will be dosed with anti-CD117 (clone ACK2) and anti-CD47 (clone MIAP410) antibodies to eliminate murine hematopoietic stem cells (HSC). Anti-CD47 antibody will be dosed at 1-50 mg/kg daily intraperitoneally for 4-10 days. Anti-CD117 antibody will be delivered intravascularly at a dose of 0.1-50 mg/kg once. Some mice will also receive one or more additional antibodies targeting CD133, CD123, CD110, CD34, CD50, CD33, CD45, or CD90 at 0.01-100 mg/kg intraperitoneally for 1-5 days.

### Depletion

HSC depletion in the bone marrow of mice treated with the additional antibody(ies) will be compared to those treated with anti-CD117 and anti-CD47 alone. Mice will be euthanized 5-21 days after final antibody treatment and bone marrow will be harvested. Flow cytometry analysis will be performed to determine the proportion of depleted cells for each lineage. Briefly, bone marrow cells will be stained with an antibody cocktail containing the following antibodies: CD45, CD11b, CD3, CD19, Ter119, Lineage cocktail, Sca-1, c-KIT, CD48, CD150. Viability dyes will also be included in the analysis.

### Engraftment (Mice)

Upon confirmation of depletion, engraftment experiments will be performed. Briefly, fully immunocompetent C57Bl/6 CD45.1/CD45.2 adult mice will be treated with anti-CD117 and anti-CD47 with and without additional antibody(ies) as described above. Following completion of the antibody dosing, 0-28 days later, mice will be injected with 0.5-10e6 lineage negative or whole bone marrow cells from C57Bl/6 CD45.2 animals. In some groups, lineage negative bone marrow cells may be transduced with a lentiviral vector with or without a transgene. Control groups will include animals not treated with the additional antibody(ies) as well as animals receiving conditioning via irradiation (400-1000 cGy) or busulfan (4 doses of 25 mg/kg). A time course of transplant following antibody dosing may be performed.

Engraftment efficiency will be measured by flow cytometry in the peripheral blood and bone marrow for the CD45 variants for 4-24 weeks after transplant. In the cases where the transplanted cells have been modified by a lentiviral vector, relevant flow cytometric and molecular assays including qPCR for vector presence will be completed to determine the level of gene marking in transplanted animals. Engraftment efficiency in the peripheral blood and bone marrow of mice treated with and without the additional antibody(ies) will be compared to determine whether engraftment levels are enhanced.

### Engraftment (Non-Human Primate)

Experiments similar to the ones above will be performed in a non-human primate model of autologous gene therapy. Briefly, rhesus macaques or other relevant non-human primates will be treated with anti-CD117 and anti-CD47 antibodies to eliminate hematopoietic stem cells (HSCs). Anti-CD47 antibody will be dosed at 1-50 mg/kg for 1-30 days. Anti-CD117 antibody will be delivered at a dose of 0.1-50 mg/kg once. The primates will also receive one or more antibodies targeting CD133, CD123, CD110, CD34, CD50, CD33, CD45, or CD90 at 0.01-100 mg/kg. Following this conditioning regimen, animals will be infused with 5-10e6 cell/kg of gene-modified autologous cells (possibly marked with a lentiviral vector for tracking). Bone marrow and peripheral blood will be sampled from the animals at one, two, four, and eight weeks as well as three, six, nine, and twelve months to monitor engraftment of the gene modified cells. Engraftment efficiency will be determined based on the molecular presence of the vector (by qPCR) and normalized based on the percentage of marked cells in the graft at the time of infusion. Engraftment efficiency in animals receiving the additional antibody(ies) will be compared to those receiving the anti-CD117 and anti-CD47 antibody cocktail alone or historical controls conditioned with total body irradiation.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for hematopoietic stem cell transplantation (HSCT) or HSC-based gene therapy in a subject comprising:
(a) administering to the subject, a first antibody, and optionally a second antibody, in a dose effective to ablate or decrease hematopoietic stem cells in the bone marrow of the subject;
(b) administering to the subject, an amount of an endopeptidase effective to degrade or digest and/or inhibit or reduce effector function of the first and second antibodies; and
(c) administering to the subject, donor hematopoietic stem cells.

2. The method of claim 1, wherein the first antibody binds an antigen expressed on a hematopoietic stem cell.

3. The method of claim 1 or claim 2, wherein the first antibody binds an antigen selected from the group consisting of: c-kit (CD117), CD133, CD34, CD50, CD33, CD45, CD123, CD110, and CD90.

4. The method of any one of claims 1 to 3, wherein the first antibody is conjugated to a cytotoxic agent.

5. The method of any one of claims 1 to 4, wherein the first antibody is conjugated to a cytotoxic agent selected from the group consisting of: a toxin, a radioisotope, an RNA polymerase II inhibitor and/or RNA polymerase III inhibitor, and a DNA-damaging agent.

6. The method of any one of claims 1 to 5, wherein the second antibody is an anti-CD47 antibody or an anti-SIRPα antibody.

7. The method of any one of claims 1 to 6, wherein the endopeptidase is a cysteine protease or a thiol protease.

8. The method of any one of claims 1 to 7, wherein the endopeptidase is isolated from Streptococcus pyogenes, Streptococcus equi, Streptococcus agalactiae, Streptococcus pseudoporcinus, Streptococcus suis, Streptococcus porcinus, Mycoplasma canis.

9. The method of any one of claims 1 to 8, wherein the endopeptidase comprises an IgG degrading enzyme (IgdE).

10. The method of any one of claims 1 to 9, wherein the endopeptidase comprises an IgdE selected from the group consisting of: an Immunoglobulin G-degrading enzyme of S. pyogenes (IdeS), Immunoglobulin G-degrading enzyme of S. equi ssp. zooepidemicus (IdeZ), an Immunoglobulin G-degrading enzyme of M. canis (IdeMC), and variants thereof.

11. A method for hematopoietic stem cell transplantation (HSCT) or adoptive cell therapy (ACT) in a subject comprising:
(a) administering to the subject, a first antibody, wherein the first antibody binds an antigen expressed on hematopoietic stem cells;
(b) administering to the subject, a second antibody, wherein the second antibody blocks CD47 binding to SIRPα;
wherein the first antibody and the second antibody are each administered in a dose effective to ablate hematopoietic stem cells in the bone marrow of the subject;
(c) administering to the subject, an amount of an endopeptidase effective to degrade or digest and/or inhibit or reduce effector function of the first and second antibodies; and
(d) administering to the subject, donor hematopoietic stem cells.

12. The method of claim 11, wherein the first antibody binds an antigen selected from the group consisting of: c-kit (CD117), CD133, CD34, CD50, CD33, CD45, CD123, CD110, and CD90.

13. The method of claim 11 or claim 12, wherein the first antibody binds CD117 (c-kit).

14. The method of claim 11 or claim 12, wherein the first antibody binds CD133.

15. The method of claim 11 or claim 12, wherein the first antibody binds CD34 or CD33.

16. The method of claim 11 or claim 12, wherein the first antibody binds CD50 or CD45.

17. The method of claim 11 or claim 12, wherein the first antibody binds CD123.

18. The method of claim 11 or claim 12, wherein the first antibody binds CD110.

19. The method of claim 11 or claim 12, wherein the first antibody binds CD90.

20. The method of any one of claims 11 to 19, wherein the first antibody is conjugated to a cytotoxic agent.

21. The method of any one of claims 11 to 20, wherein the first antibody is conjugated to a cytotoxic agent selected from the group consisting of: a toxin, a radioisotope, an RNA polymerase II inhibitor and/or RNA polymerase III inhibitor, and a DNA-damaging agent.

22. The method of any one of claims 11 to 21, wherein the second antibody is an anti-CD47 antibody or an anti-SIRPα antibody.

23. The method of any one of claims 11 to 22, wherein the second antibody is an anti-CD47 antibody.

24. The method of any one of claims 11 to 22, wherein the second antibody is an anti-SIRPα antibody.

25. The method of any one of claims 11 to 24, wherein the endopeptidase is a cysteine protease or a thiol protease.

26. The method of any one of claims 11 to 25, wherein the endopeptidase is isolated from Streptococcus pyogenes, Streptococcus equi, Streptococcus agalactiae, Streptococcus pseudoporcinus, Streptococcus suis, Streptococcus porcinus, Mycoplasma canis.

27. The method of any one of claims 11 to 26, wherein the endopeptidase comprises an IgG degrading enzyme (IgdE).

28. The method of any one of claims 11 to 27, wherein the endopeptidase comprises an IgdE selected from the group consisting of: an Immunoglobulin G-degrading enzyme of S. pyogenes (IdeS), Immunoglobulin G-degrading enzyme of S. equi ssp. zooepidemicus (IdeZ), an Immunoglobulin G-degrading enzyme of M. canis (IdeMC), and variants thereof.

29. A method for hematopoietic stem cell transplantation (HSCT) or adoptive cell therapy (ACT) in a subject comprising:
(a) administering a first antibody to the subject, wherein the first antibody is an anti-CD117 antibody;
(b) administering a second antibody to the subject, wherein the second antibody is an anti-CD47 or anti-SIRPα antibody blocks CD47 binding to SIRPα;
wherein the first antibody and the second antibody are each administered in a dose effective to ablate hematopoietic stem cells in the bone marrow of the subject;
(c) administering to the subject, an amount of an endopeptidase effective to degrade or digest and/or inhibit or reduce effector function of the first antibody and the second antibody; and
(d) administering to the subject, donor hematopoietic stem cells.

30. The method of claim 29, wherein the first antibody is conjugated to a cytotoxic agent.

31. The method of claim 29 or claim 30, wherein the first antibody is conjugated to a cytotoxic agent selected from the group consisting of: a toxin, a radioisotope, an RNA polymerase II inhibitor and/or RNA polymerase III inhibitor, and a DNA-damaging agent.

32. The method of any one of claims 29 to 31, wherein the second antibody is an anti-CD47 antibody.

33. The method of any one of claims 29 to 32, wherein the second antibody is an anti-SIRPα antibody.

34. The method of any one of claims 29 to 33, wherein the endopeptidase is a cysteine protease or a thiol protease.

35. The method of any one of claims 29 to 34, wherein the endopeptidase is isolated from Streptococcus pyogenes, Streptococcus equi, Streptococcus agalactiae, Streptococcus pseudoporcinus, Streptococcus suis, Streptococcus porcinus, Mycoplasma canis.

36. The method of any one of claims 29 to 35, wherein the endopeptidase comprises an IgG degrading enzyme (IgdE).

37. The method of any one of claims 29 to 36, wherein the endopeptidase comprises an IgdE selected from the group consisting of: an Immunoglobulin G-degrading enzyme of S. pyogenes (IdeS), Immunoglobulin G-degrading enzyme of S. equi ssp. zooepidemicus (IdeZ), an Immunoglobulin G-degrading enzyme of M. canis (IdeMC), and variants thereof.

38. A method for hematopoietic stem cell transplantation (HSCT) or adoptive cell therapy (ACT) in a subject comprising:
(a) administering a first antibody to the subject, wherein the first antibody is an anti-CD117 antibody;
(b) administering a second antibody to the subject, wherein the first antibody is an anti-CD47 antibody that blocks CD47 binding to SIRPα;
wherein the first antibody and the second antibody are each administered in a dose effective to ablate hematopoietic stem cells in the bone marrow of the subject;
(c) administering to the subject, an amount of a cysteine protease effective to degrade or digest and/or inhibit or reduce effector function of the first and second antibodies, wherein the protease or glycosidase is a cysteine protease; and
(d) administering to the subject, donor hematopoietic stem cells.

39. The method of claim 38, wherein the first antibody is conjugated to a cytotoxic agent.

40. The method of claim 38 or claim 39, wherein the first antibody is conjugated to a cytotoxic agent selected from the group consisting of: a toxin, a radioisotope, an RNA polymerase II inhibitor and/or RNA polymerase III inhibitor, and a DNA-damaging agent.

41. The method of any one of claims 38 to 40, wherein the cysteine protease is isolated from Streptococcus pyogenes, Streptococcus equi, Streptococcus agalactiae, Streptococcus pseudoporcinus, Streptococcus suis, Streptococcus porcinus, Mycoplasma canis.

42. The method of any one of claims 38 to 41, wherein the cysteine protease comprises an IgG degrading enzyme (IgdE).

43. The method of any one of claims 38 to 42, wherein the cysteine protease comprises an IgdE selected from the group consisting of: an Immunoglobulin G-degrading enzyme of S. pyogenes (IdeS), Immunoglobulin G-degrading enzyme of S. equi ssp. zooepidemicus (IdeZ), an Immunoglobulin G-degrading enzyme of M. canis (IdeMC), and variants thereof.

44. The method of any one of claims 1 to 43, wherein the first antibody and the second antibody are administered to the subject at about the same time.

45. The method of any one of claims 1 to 43, wherein the first antibody is administered to the subject before the second antibody is administered to the subject.

46. The method of any one of claims 1 to 43, wherein the first antibody is administered to the subject after the second antibody is administered to the subject.

47. The method of any one of claims 1 to 46, wherein the endopeptidase is administered to the subject after both the first antibody and the second antibody have been administered to the subject.

48. The method of any one of claims 1 to 47, wherein the endopeptidase is administered to the subject at least 3 days, at least 4 days, at least 5 days, at least 6 days, at least 7 days, at least 8 days, at least 9 days, at least 10 days, at least 11 days, at least 12 days, at least 13 days, or at least 14 days after the day that the last of the first antibody and the second antibody have been administered to the subject.

49. The method of any one of claims 1 to 48, wherein the donor hematopoietic stem cells are administered to the subject after the endopeptidase is administered to the subject.

50. The method of any one of claims 1 to 49, wherein the donor hematopoietic stem cells are administered to the subject at least 3 days, at least 4 days, at least 5 days, at least 6 days, at least 7 days, at least 8 days, at least 9 days, at least 10 days, at last 11 days, at least 12 days, at least 13 days, or at least 14 days after the endopeptidase is administered to the subject.

51. The method of any one of claims 1 to 50, wherein the donor hematopoietic stem cells are administered to the subject no more than 21 days, no more than 20 days, no more than 19 days, no more than 18 days, no more than 17 days, no more than 16 days, no more than 15 days, no more than 14 days, no more than 13 days, no more than 12 days, no more than 11 days, no more than 10 days, no more than 9 days, no more than 8 days, no more than 7 days, no more than 6 days, or no more than 5 days after the endopeptidase is administered to the subject.

52. The method of any one of claims 1 to 51, wherein the donor hematopoietic stem cells are allogenic to the subject.

53. The method of any one of claims 1 to 52, wherein the donor hematopoietic stem cells are autologous to the subject.

54. The method of any one of claims 1 to 53, wherein the donor hematopoietic stem cells comprise a vector comprising a polynucleotide that encodes a therapeutic polypeptide.

55. The method of any one of claims 1 to 53, wherein the donor hematopoietic stem cells comprise one or more genome edits.

56. The method of any one of claims 1 to 53, wherein the donor hematopoietic stem cells comprise a vector comprising a polynucleotide that encodes a therapeutic polypeptide and one or more genome edits.

57. The method of any one of claims 1 to 53, wherein the donor hematopoietic stem cells comprise a cell-based gene therapy.

58. The method of any one of claims 1 to 57, wherein the subject has a disease amenable to treatment with a cell-based gene therapy.

59. The method of any one of claims 1 to 58, wherein the subject has a disease selected from the group consisting of an autoimmune disease, a hemoglobinopathy, a leukodystropy, a lysosomal storage disease, and a neurogenerative disease.

60. The method of any one of claims 1 to 58, wherein the subject has a disease selected from the group consisting of ADA-SCID, Artemis protein deficiency, Batten disease, β-thalassemia, sickle cell disease, cerebral ALD, chronic granulomatous diseases, Fabry disease, Fanconi anemia, Gaucher disease, Hemophilia A, Hemophilia B, Leukocyte adhesion deficiency, metachromatic leukodystrophy, MPS I (Hurler syndrome), MPS II (Hunter's syndrome), MPS III (Sanfilippo Syndrome), MPS IV-A (Morquio A Syndrome), MPS IV-B (Morquio B Syndrome), MPS VI (Maroteaux-Lamy Syndrome), Pompe Disease (acid alpha-glucosidase); RAG deficiency, Wiskott Aldrich syndrome, X-linked lymphoproliferative syndrome, and X-Linked Severe Combined Immunodeficiency (X-SCID).

61. The method of any one of claims 1 to 60, wherein the subject has ADA-SCID and is administered a cell-based gene therapy comprising hematopoietic stem cells comprising a vector that comprises a polynucleotide encoding adenosine deaminase.

62. The method of any one of claims 1 to 60, wherein the subject has Artemis protein deficiency and is administered a cell-based gene therapy comprising hematopoietic stem cells comprising a vector that comprises a polynucleotide encoding DCLRE1C.

63. The method of any one of claims 1 to 60, wherein the subject has Batten Disease and is administered a cell-based gene therapy comprising hematopoietic stem cells comprising a vector that comprises a polynucleotide encoding CLN1, CLN2, CLN3, CLN4, CLN5, CLN6, CLN7, CLN8, or CLN10.

64. The method of any one of claims 1 to 60, wherein the subject has CALD and is administered a cell-based gene therapy comprising hematopoietic stem cells comprising a vector that comprises a polynucleotide encoding ATP-binding cassette, sub-family D, member 1 (ABCD1).

65. The method of any one of claims 1 to 60, wherein the subject has a chronic granulomatous disease and is administered a cell-based gene therapy comprising hematopoietic stem cells comprising a vector that comprises a polynucleotide encoding cytochrome b-245 alpha chain, cytochrome b-245 beta chain, neutrophil cytosolic factor 1, neutrophil cytosolic factor 2, or neutrophil cytosolic factor 4.

66. The method of any one of claims 1 to 60, wherein the subject has Fabry disease and is administered a cell-based gene therapy comprising hematopoietic stem cells comprising a vector that comprises a polynucleotide encoding alpha-galactosidase A.

67. The method of any one of claims 1 to 60, wherein the subject has Fanconi anemia and is administered a cell-based gene therapy comprising hematopoietic stem cells comprising a vector that comprises a polynucleotide encoding FANCA, FANCC, or FANCG.

68. The method of any one of claims 1 to 60, wherein the subject has Gaucher disease and is administered a cell-based gene therapy comprising hematopoietic stem cells comprising a vector that comprises a polynucleotide encoding glucocerebrosidase.

69. The method of any one of claims 1 to 60, wherein the subject has Hemophilia A and is administered a cell-based gene therapy comprising hematopoietic stem cells comprising a vector that comprises a polynucleotide encoding Factor VIII.

70. The method of any one of claims 1 to 60, wherein the subject has Hemophilia B and is administered a cell-based gene therapy comprising hematopoietic stem cells comprising a vector that comprises a polynucleotide encoding Factor IX.

71. The method of any one of claims 1 to 60, wherein the subject has Leukocyte adhesion deficiency and is administered a cell-based gene therapy comprising hematopoietic stem cells comprising a vector that comprises a polynucleotide encoding CD18.

72. The method of any one of claims 1 to 60, wherein the subject has MLD and is administered a cell-based gene therapy comprising hematopoietic stem cells comprising a vector that comprises a polynucleotide encoding arylsulfatase A.

73. The method of any one of claims 1 to 60, wherein the subject has MPS I and is administered a cell-based gene therapy comprising hematopoietic stem cells comprising a vector that comprises a polynucleotide encoding alpha-L-iduronidase.

74. The method of any one of claims 1 to 60, wherein the subject has MPS II and is administered a cell-based gene therapy comprising hematopoietic stem cells comprising a vector that comprises a polynucleotide encoding iduronate 2-sulfatase.

75. The method of any one of claims 1 to 60, wherein the subject has MPS III and is administered a cell-based gene therapy comprising hematopoietic stem cells comprising a vector that comprises a polynucleotide encoding N-sulfoglucosamine sulfohydrolase.

76. The method of any one of claims 1 to 60, wherein the subject has MPS IV-A and is administered a cell-based gene therapy comprising hematopoietic stem cells comprising a vector that comprises a polynucleotide encoding galactosamine-6 sulfatase.

77. The method of any one of claims 1 to 60, wherein the subject has MPS IV-B and is administered a cell-based gene therapy comprising hematopoietic stem cells comprising a vector that comprises a polynucleotide encoding beta-galactosidase.

78. The method of any one of claims 1 to 60, wherein the subject has MPS VI and is administered a cell-based gene therapy comprising hematopoietic stem cells comprising a vector that comprises a polynucleotide encoding N-acetylgalactosamine-4-sulphatase.

79. The method of any one of claims 1 to 60, wherein the subject has Pompe Disease and is administered a cell-based gene therapy comprising hematopoietic stem cells comprising a vector that comprises a polynucleotide encoding acid alpha-glucosidase

80. The method of any one of claims 1 to 60, wherein the subject has a RAG deficiency and is administered a cell-based gene therapy comprising hematopoietic stem cells comprising a vector that comprises a polynucleotide encoding RAG1 or RAG2.

81. The method of any one of claims 1 to 60, wherein the subject has Wiskott-Aldrich syndrome and is administered a cell-based gene therapy comprising hematopoietic stem cells comprising a vector that comprises a polynucleotide encoding Wiskott-Aldrich syndrome protein.

82. The method of any one of claims 1 to 60, wherein the subject has X-SCID and is administered a cell-based gene therapy comprising hematopoietic stem cells comprising a vector that comprises a polynucleotide encoding interleukin 2 receptor gamma.

83. The method of any one of claims 1 to 60, wherein the subject has a hemoglobinopathy and is administered a cell-based gene therapy comprising hematopoietic stem cells comprising a vector that comprises a polynucleotide encoding a globin protein.

84. The method of any one of claims 1 to 60, wherein the subject has 0-thalassemia and is administered a cell-based gene therapy comprising hematopoietic stem cells comprising a vector that comprises a polynucleotide encoding a β-globin protein, a 6-globin protein, a γ-globin protein, an anti-sickling globin, a βA-T87Q-globin protein, a βA-G16D/E22A/T87Q-globin protein, or a βA-T87Q/K95E/K120E-globin protein.

85. The method of any one of claims 1 to 60, wherein the subject has sickle cell disease and is administered a cell-based gene therapy comprising hematopoietic stem cells comprising a vector that comprises a polynucleotide encoding a β-globin protein, a δ-globin protein, a γ-globin protein, an anti-sickling globin, a βA-T87Q-globin protein, a βA-G16D/E22A/T87Q-globin protein, or a βA-T87Q/K95E/K120E-globin protein.

86. The method of any one of claims 1 to 85, wherein the method further comprises administering one or more HSC mobilization agents to the subject before step (a).

87. The method of claim 86, wherein the one or more HSC mobilization agents is selected from the group consisting of: GM-CSF, G-CSF (filgrastim), pegylated G-CSF (pegfilgrastim), glycosylated G-CSF (lenograstim), AMD3100 (plerixafor), macrophage inflammatory protein 1α (MIP1α), CCL3, SDF-1α peptide analogs (CTCE-0021, CTCE-0214), Met-SDF-1β, IL-8, GRO proteins (GRO-β (CXCL2)), SB-251353, and suitable combinations thereof.

88. The method of claim 86 or claim 87, wherein the one or more HSC mobilization agents is G-CSF (filgrastim) and/or AMD3100 (plerixafor).

89. The method of claim 86 or claim 87, wherein the one or more HSC mobilization agents is G-CSF (filgrastim) and AMD3100 (plerixafor).

90. The method of any one of claims 1 to 85, wherein the method further comprises administering one or more chemotherapeutic agents in an amount effective to increase macrophage activation and recruitment to the bone marrow of the subject.

91. The method of claim 90, wherein the one or more chemotherapeutic agents is selected from the group consisting of: cyclophosphamide, busulphan, treosulphan, melphalan, thiotepa, carboplatin, carmustine, etoposide, cytosine arabinoside (AraC), and fludarabine.

92. The method of claim 90, wherein the one or more chemotherapeutic agents is cyclophosphamide.
